(19) United States
(12) Patent Application Publication
(10) Pub. No.: US 2022/0282236 A1
(43) Pub. Date: Sep. 8, 2022

(54) CREATININE DEIMINASE AND USES THEREOF

(71) Applicant: MEON MEDICAL SOLUTIONS GMBH & CO. KG, Graz (AT)

(72) Inventor: Helmut SCHWAB, Graz (AT)

(21) Appl. No.: 17/734,169

(22) Filed: May 2, 2022

Related U.S. Application Data
(62) Division of application No. 16/955,694, filed on Jun. 18, 2020.

(30) Foreign Application Priority Data
Dec. 21, 2017 (EP) 17209848.5

(51) Int. Cl. C12N 9/78; C12Q 1/00; C12Q 1/34

(52) U.S. Cl. CPC C12N 9/78; C12Q 1/005; C12Q 1/34; C12Y 305/04021

(57) ABSTRACT

The present invention relates broadly to the field of creatinine determination. In particular, it provides a novel creatinine deiminase characterized by novel nucleic acid and amino acid sequences and superior enzymatic activity. It also provides uses of this creatinine deiminase, including assays for determining the amount of creatinine in a sample. These can be useful inter alia for the detection of kidney disease.

### CROSS-REFERENCE TO RELATED APPLICATIONS

This application is a divisional application of U.S. application Ser. No. 16/955,694, filed Jun. 18, 2020, which is a U.S. national phase of International Application No. PCT/EP2018/086215, filed on Dec. 20, 2018, which claims priority to European Patent Application No. 17209848.5, filed Dec. 21, 2017.

### FIELD OF THE INVENTION

The present invention relates broadly to the field of creatinine determination. In particular, it provides a novel creatinine deiminase characterized by novel nucleic acid and amino acid sequences and superior enzymatic activity. It also provides uses of this creatinine deiminase, including assays for determining the amount of creatinine in a sample. These can be useful inter alia for the detection of kidney disease.

### BACKGROUND OF THE INVENTION

The enzyme creatinine deiminase, also named creatinine amidohydrolase (EC 3.5.4.21) catalyzes the hydrolysis of creatinine to N-methylhydantoin, thereby releasing ammonia. It is involved in bacterial metabolism for degradation of creatinine, and of interest for diagnostic determination of creatinine in urine and serum. Creatinine deiminase is a metalloprotein and Zn²⁺ and Fe²⁺ are efficient in its activation. The 3-D structure of a related enzyme, cytosine deaminase, revealed that Zn²⁺ and Fe²⁺ ions are present in the active site as catalytic metal ions in such enzymes. Based on this structure, it was also possible to generate a 3-D structure model of the creatinine deiminase from Tissierella creatinini, the protein sequence of which is described as SEQ ID NO: 1 in EP 1 325 958 A1. In this structure model, a Zn²⁺ atom is positioned at the active site (Nishiya, Int J Anal Bio-Sci, 1: 55-59, 2013).

The creatinine deiminase from T. creatinini is known to have superior properties with respect to applications in the diagnostic analysis of creatinine. EP 1 325 958 A1 describes the molecular cloning and recombinant expression of this enzyme. However, as of now, to the inventor's best knowledge, no reports on the recombinant production or use of the recombinant enzyme of EP 1 325 958 A1 exist in the scientific or the patent literature.

The inventor has attempted to establish a recombinant production process for the creatinine deiminase according to the sequence information and expression setup described in EP 1 325 958 A1. All attempts failed, however, since the teachings of EP 1 325 958 A1 led to an enzymatically inactive creatinine deiminase protein. The inventor then found that creatinine deiminase nucleotide and amino acid sequences different from those taught in EP 1 325 958 A1 result in an active protein. In particular, the inventor found in expression and activity analysis experiments that untranslated regions enable or facilitate the expression of active T. creatinini creatinine deiminase. Sufficiently high expression levels are paramount to the commercialization of enzyme-based applications.

Furthermore, the inventor found that the use of Mn²⁺ as a catalytic metal ion increases activity and stability of creatinine deiminase. Under Mn²⁺ loading the enzyme shows a superior behaviour when compared to commercially available creatinine deiminase.

### SUMMARY OF THE INVENTION

In a first aspect, the present invention relates to an isolated creatinine deiminase polypeptide comprising an amino acid sequence according to SEQ ID NO: 4 or an at least 80% sequence identity variant thereof, wherein the isolated creatinine deiminase polypeptide has creatinine deiminase activity.

In a second aspect, the present invention relates to an isolated nucleic acid encoding for a creatinine deiminase polypeptide as defined in the first aspect.

In a third aspect, the present invention relates to a vector comprising the nucleic acid of the second aspect.

In a fourth aspect, the present invention relates to a cell comprising the polypeptide of the first aspect, the nucleic acid of the second aspect, or the vector of the third aspect.

In a fifth aspect, the present invention relates to a method for producing a creatinine deiminase polypeptide as defined in the first aspect, comprising the steps of

(i) expressing the nucleic acid as defined in the second aspect in a cell as defined in the fourth aspect, and
(ii) isolating the creatinine deiminase polypeptide.

In a sixth aspect, the present invention relates to a creatinine deiminase polypeptide produced with the method as defined in the fifth aspect.

In a seventh aspect, the present invention relates to the use of a creatinine deiminase polypeptide as defined in the first or sixth aspect for determining the amount of creatinine in a sample.

In an eighth aspect, the present invention relates to a kit suitable for determining the amount of creatinine in a sample, comprising the creatinine deiminase polypeptide as defined in the first or the sixth aspect.

In a ninth aspect, the present invention relates to a composition suitable for determining the amount of creatinine in a sample, comprising the creatinine deiminase polypeptide as defined in the first or the sixth aspect.

In a tenth aspect, the present invention relates to an in vitro method for detecting kidney disease in a subject, comprising determining the amount of creatinine in a sample from the subject as defined in the seventh aspect, wherein an amount of creatinine that is larger than the normal value indicates that the subject has kidney disease.

### LEGENDS TO THE FIGURES

FIG. 1: Cloning strategies for expression of creatinine deiminase (explanations see Example 2). A: Strategy 1; B: Strategy 2.

FIG. 2: SDS Page analysis of whole cell lysates of expression clones. It can be clearly seen that the protein from clones derived from the synthetic DNA is migrating faster in comparison to the protein from clones derived from the genomic DNA. All clones of strategy 2 did not express visible amounts of creatinine deiminase protein.

| Lane | Clone no | Temp | Construct |
|---|---|---|---|
| 1 | 1 | 28° C. | Genomic (Seq ID 3), strategy 1, |
| | | | orientation 1 |
| 2 | 1 | 25° C. | Genomic (Seq ID 3), strategy 1, |
| | | | orientation 1 |
| 3 | 2 | 28° C. | Genomic(Seq ID 3), strategy 1, |
| | | | orientation 2 |
| 4 | 2 | 25° C. | Genomic (Seq ID 3), strategy 1, |
| | | | orientation 2 |
| 5 | 8 | 28° C. | Synthetic (Seq ID 2), strategy 1, |
| | | | orientation 1 |
| 6 | 8 | 25° C. | Synthetic (Seq ID 2), strategy 1, |
| | | | orientation 1 |
| 7 | 9 | 28° C. | Synthetic (Seq ID 2), strategy 1, |
| | | | orientation 2 |
| 8 | 9 | 25° C. | Synthetic (Seq ID 2), strategy 1, |
| | | | orientation 2 |
| 9 | 17 | 28° C. | Synthetic (Seq ID 2), strategy 2, |
| | | | C-terminal His tag |
| 10 | 17 | 25° C. | Synthetic (Seq ID 2), strategy 2, |
| | | | C-terminal His tag |
| 11 | EVC | 28° C. | Empty vector control (pMS470) |
| 12 | standard | | 5 ul page ruler size standard |
| 13 | 21 | 28° C. | Synthetic (Seq ID 2), strategy 2, |
| | | | N-terminal His tag |
| 14 | 21 | 25° C. | Synthetic (Seq ID 2), strategy 2, |
| | | | N-terminal His tag |
| 15 | 13 | 28° C. | Synthetic (Seq ID 2), strategy 2, |
| | | | untagged |

FIG. 3: SDS Page analysis of protein fractions obtained by centrifugal fractionation.

| Lane | Clone no | Protein fraction | Construct |
|---|---|---|---|
| 1 | 1 | Total cell lysate | Genomic (Seq ID 3) |
| 2 | 1 | 3000 × g pellet | Genomic (Seq ID 3) |
| 3 | 1 | 3000 × g supernatant | Genomic(Seq ID 3) |
| 4 | 1 | 16000 × g pellet | Genomic (Seq ID 3) |
| 5 | 8 | 16000 × g supernatant | Genomic (Seq ID 3) |
| 6 | 8 | Total cell lysate | Synthetic (Seq ID 2) |
| 7 | g | 3000 × g pellet | Synthetic (Seq ID 2) |
| 8 | 8 | 3000 × g supernatant | Synthetic (Seq ID 2) |
| 9 | 8 | 16000 × g pellet | Synthetic (Seq ID 2) |
| 10 | 8 | 16000 × g supernatant | Synthetic (Seq ID 2) |
| 11 | standard | | 5 ul page ruler size standard |
| 12 | EVC | 3000 × g pellet | Empty vector control (pMS470) |
| 13 | EVC | 3000 × g supernatant | Empty vector control (pMS470) |
| 14 | EVC | 16000 × g pellet | Empty vector control (pMS470) |
| 15 | EVC | 16000 × g supernatant | Empty vector control (pMS470) |

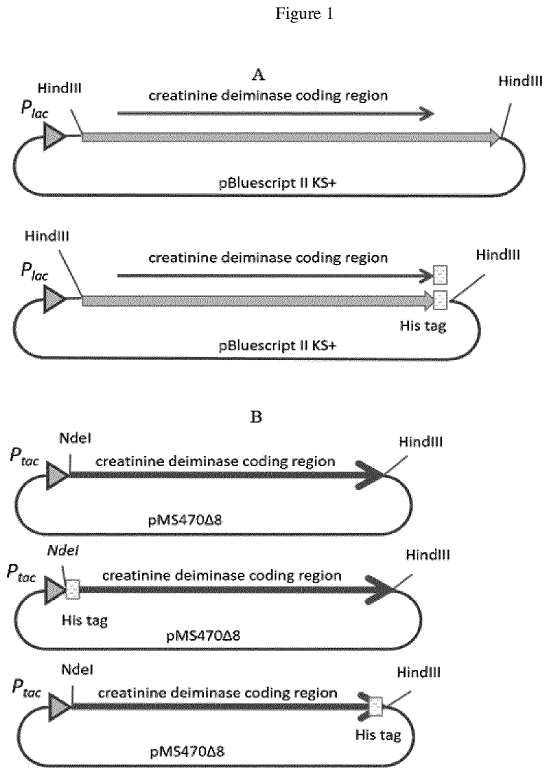

FIG. 4: Activity analysis of clones. a: clone 1, 16000 g supernatant, b: ctHis no creatinine, c: ctHis purified 1:10, d: Clone 1, no creatinine, e: ctHis 16000 g supernatant, f: Clone 8, 16000 g supernatant. Clone 1: genomic fragment, strategy 1, orientation 1; clone 8: synthetic fragment, strategy 1, orientation 1; ctHis: genomic fragment, strategy 1, orientation 1, C-terminal His tag inserted (see FIG. 1). ctHis purified: protein purified by Ni-chelate chromatography.

FIG. 5: SDS PAGE analysis of pellet and supernatant fractions of Hs-tagged variants. The non-tagged variants clone 1 and clone 8 were taken as reference. The activity of the lysates was semi-quantitatively estimated form the slopes of the NADH consumption. For constructs refer to FIG. 1 (clones 1 and 8) and FIG. 6 (clones 3h, 4H, 6h and 11h); n.d.: not determined

| | | | | Creatinine |
|---|---|---|---|---|
| Lane | Clone no | Protein fraction | Construct | deiminase activity |
| 1 | 8 | 16,000 × g pellet | Synthetic, no tag | n.d. |
| 2 | 8 | 16,000 × g supernatant | Synthetic, no tag | no |
| 3 | 1 | 16,000 × g pellet | Genomic, no tag, | n.d. |
| 4 | 1 | 16,000 × g supernatant | Genomic, no tag, | ++++ |
| 5 | 4h | 16,000 × g pellet | Genomic, C-long, 3′ | n.d. |
| 6 | 4h | 16,000 × g supernatant | Genomic, C-long, 3′ | + |
| 7 | 3h | 16,000 × g supernatant | Genomic, C-short, 3′ | + |
| 8 | 3h | 16,000 × g pellet | Genomic, C-short, 3′ | n.d. |
| 9 | standard | | page ruler size standard | |
| 10 | 1h | 16,000 × g supernatant | Genomic, C-long, 3′ & 5′ | +++ |
| 11 | 1h | 16,000 × g pellet | Genomic, C-long, 3′ & 5′ | n.d. |
| 12 | 6h | 16,000 × g supernatant | Genomic, C-short, 3′ & 5′ | +++ |
| 13 | 6h | 16,000 × g pellet | Genomic, C-short, 3′ & 5′ | n.d. |
| 14 | 11h | 16,000 × g supernatant | Genomic, N-long, 3′ & 5′ | +++ |
| 15 | 11h | 16,000 × g pellet | Genomic, N-long, 3′ & 5′ | n.d. |

FIG. 6: SDS PAGE of lysate and elution fractions from Ni-chelate chromatography purification of clone 1h. Pellet and supernatant fractions of untagged clones 8 and 1, respectively were loaded as reference.

| Lane | |
|---|---|
| 1 | Clone 8 16000 g pellet |
| 2 | Clone1 16000 g SN |
| 2 | Crude lysate (sonicated) |
| 4 | 16,000 × g pellet |
| s | 16,000 × g supernatant |
| 6 | 16,000 × g supernatant, sterile filtered |
| 7 | Flowtrough |
| 8 | Wash |
| 9 | Elution fraction 1 |
| 10 | Elution fraction 2 |
| 11 | Elution fraction 3 |
| 12 | Elution fraction 4 |
| 13 | Elution fraction 5 |
| 14 | Standard |
| 15 | Polled elution fractions F1-F3 desalted |

FIG. 7: SDS Page of creatinine deiminase protein preparations. Based on the determination of the protein concentration, 0.7 μg protein were loaded of each protein (clones 1 and 11 were used as references). Clone 1 (lane 2) was applied as 16,000×g supernatant preparation, the clones 1h and 11h were applied as Ni-chelate purified protein preparations. As it is not known what is present in the Toyobo enzyme preparation as stabilizer, the enzyme amount for preparation of the solution was weighted in.

| | | | μg protein |
|---|---|---|---|
| lane | Enzyme | Construct | loaded on gel |
| 2 | Clone 1 | Genome, no tag | 0.7 μg |
| 3 | standard | | 0.7 μg |
| 4 | Clone 11h | Genome, N-long, 5′ & 3′ | 0.7 μg |
| 5 | Toyobo | purchased prearation | 0.7 μg |
| 6 | Clone 1h | Genome, C-long, 5′ & 3′ | 0.7 μg |

FIG. 8: Reaction curves from activity assays with creatinine deiminases from T. creatintini and a commercial preparation of Toyobo. For details see Example 5. A: 1 μg creatinine deiminase protein. B: 0.1 μg creatinine deiminase protein. a: blank, b: Toyobo, c: clone 1h, d: clone 11h

FIG. 9: SDS PAGE analysis of samples from the stability test. 20 μg protein of each sample was loaded onto the gel.

| Lane | Sample |
|---|---|
| 3 | Storage at 20° C., thawn (day 0) |
| 2 | Storage at 4° C., 25 days |
| 3 | Storage at 23° C., 25 days |
| 4 | Storage at 37° C., 25 days |

FIG. 10: Creatinine determination with creatinine deiminase from T. creatinini. The ΔE values from 2 independent determinations and taken from 10 min (triangles and squares) and 20 min (dots and crosses) reaction time.

FIG. 11: SDS gel analysis of lysate and purified protein. Lanes 1 & 5; Size standard (Page ruler pre-stained protein ladder); Lane 2: cell lysate; Lane 3: 2 μg purified protein; Lane 4: 5 μg purified protein.

FIG. 12: Specific activity of metal-loaded protein preparations (Cdi-metal exchange). WT: untreated protein (produced with Mn²⁺ added to the medium); Mn, Mn+Fe, Mn+Zn, F+Zn and Mn+Fe+Zn: Apo protein (metal-extracted preparation of WT) treated with the respective metal²⁺ ion(s).

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturers' specifications, instructions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments, which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, are to be understood to imply the inclusion of a stated integer or step or group of integers or steps but not the exclusion of any other integer or step or group of integer or step. As used in this specification and the appended claims, the singular forms “a”, “an”, and “the” include plural referents, unless the content clearly dictates otherwise.

### Aspects of the Invention and Particular Embodiments Thereof

In a first aspect, the present invention relates to an isolated creatinine deiminase polypeptide comprising an amino acid sequence according to SEQ ID NO: 4 or an at least 80% sequence identity variant thereof, wherein the isolated creatinine deiminase polypeptide has creatinine deiminase activity.

References herein to the isolated creatinine deiminase polypeptide include the polypeptide comprising an amino acid sequence according to SEQ ID NO: 4, as well as the variant thereof.

According to the first aspect, the isolated creatinine deiminase polypeptide is enzymatically active. The activity of the creatinine deiminase polypeptide comprising an amino acid sequence according to SEQ ID NO: 4 is preferably at least 10 U/mg, more preferably at least 15 U/mg at a concentration of 0.002 to 0.02 (e.g. 0.005) mg/ml creatinine deiminase polypeptide, or at least 20 U/mg, more preferably at least 24 U/mg at a concentration of 0.002 mg/ml creatinine deiminase polypeptide. The variant has at least 50% of the enzyme activity of the isolated creatinine deiminase polypeptide, preferably at least 60%, more preferably at least 70%, at least 80%, at least 90% or at least 95%, most preferably the same activity (ideally 100%).

The isolated creatinine deiminase polypeptide is preferably soluble.

In a preferred embodiment, the sequence of the variant retains, regardless of the minimum level of sequence identity, one or more of the following:

amino acid 364 of SEQ ID NO: 4,

amino acid 371 of SEQ ID NO: 4,

amino acid 394 of SEQ ID NO: 4, and/or

one or more, preferably all, of amino acid residues 410 to 419 of SEQ ID NO: 4.

In another preferred embodiment, the isolated creatinine deiminase polypeptide is bound to or is capable of binding at least one metal dication, for example selected from the group consisting of Zn²⁺, Fe²⁺, Ni²⁺ and Mn²⁺, preferably Mn²⁺. In one embodiment, it is bound to Mn²⁺, and optionally also Zn²⁺ or Fe²⁺. Of the optional Zn²⁺ or Fe²⁺, Zn²⁺ is preferred.

As implied by the fifth and sixth aspect below, the invention also relates to a plurality of creatinine deiminase polypeptides of the first aspect. In a particular embodiment, the molar ratio of Mn²⁺ to creatinine deiminase polypeptide of the plurality of the creatinine deiminase polypeptides is at least 0.05 or 0.10 (metal:protein subunit), preferably at least 0.15 (e.g. at least 0.17, 0.19, 0.21 or 0.23), and more preferably at least 0.25. The theoretical upper limit for Mn²⁺ according to the invention is 2 (a metal dication can bind to each of the two protein subunits), which can be combined with each of the afore-mentioned lower limits. In one preferred embodiment, the upper limit for Mn²⁺ is 1, which can also be combined with each of the afore-mentioned lower limits. In another preferred embodiment, the upper limit for Mn²⁺ is 0.5, which can also be combined with each of the afore-mentioned lower limits.

Preferably, in this particular embodiment, the molar ratio of Zn²⁺ to creatinine deiminase polypeptide of the plurality of the creatinine deiminase polypeptides is at least 0.05 or 0.15 (metal:protein subunit), preferably at least 0.25 (e.g. at least 0.3, 0.4 or 0.5), and most preferably at least 0.52. The theoretical upper limit for Zn²⁺ according to the invention is 2 minus the minimum ratio selected for Mn²⁺, e.g. 1.95 if the minimum ratio of Mn²⁺ is 0.05, or, if Fe²⁺ is also comprised (see below), it is 2 minus the minimum ratio selected for Mn²⁺ and minus the minimum ratio selected for Fe²⁺. This can be combined with each of the afore-mentioned Zn²⁺ lower limits. In one preferred embodiment, the upper limit for Zn²⁺ is 1.6, which can also be combined with each of the afore-mentioned lower limits. In another preferred embodiment, the upper limit for Zn²⁺ is 1.3, which can also be combined with each of the afore-mentioned lower limits.

Also envisaged, in this particular embodiment, is that the molar ratio of Fe²⁺ to creatinine deiminase polypeptide of the plurality of the creatinine deiminase polypeptides is at least 0.05 or 0.10 (metal:protein subunit), preferably at least 0.15 (e.g. at least 0.2, 0.25 or 0.3), and most preferably at least 0.42. The theoretical upper limit for Fe²⁺ according to the invention is 2 minus the minimum ratio selected for Mn²⁺, e.g. 1.95 if the minimum ratio of Mn²⁺ is 0.05, or, if Zn²⁺ is also comprised (see above), it is 2 minus the minimum ratio selected for Mn²⁺ and minus the minimum ratio selected for Zn²⁺. This can be combined with each of the afore-mentioned Fe²⁺ lower limits. In one preferred embodiment, the upper limit for Fe²⁺ is 1.2, which can also be combined with each of the afore-mentioned lower limits. In another preferred embodiment, the upper limit for Fe²⁺ is 0.7, which can also be combined with each of the afore-mentioned lower limits.

In various preferred embodiments, the molar ratios to creatinine deiminase polypeptide of the plurality of the creatinine deiminase polypeptides are as follows (metal:protein subunit; in each embodiment, the upper limit for the molar ratio is preferably 1 for Mn²⁺ and 1.5 for Zn²⁺):

Mn²⁺: at least 0.05 and Zn²⁺ at any of the above ratios (and optionally Fe²⁺ at no more than 1);
Mn²⁺: at least 0.10 and Zn²⁺ at any of the above ratios (and optionally Fe²⁺ at no more than 1);
Mn²⁺: at least 0.15 and Zn²⁺ at any of the above ratios (and optionally Fe²⁺ at no more than 1);
Mn²⁺: at least 0.17 and Zn²⁺ at any of the above ratios (and optionally Fe²⁺ at no more than 1);
Mn²⁺: at least 0.19 and Zn²⁺ at any of the above ratios (and optionally Fe²⁺ at no more than 1);
Mn²⁺: at least 0.21 and Zn²⁺ at any of the above ratios (and optionally Fe²⁺ at no more than 1);
Mn²⁺: at least 0.23 and Zn²⁺ at any of the above ratios (and optionally Fe²⁺ at no more than 1);
Mn²⁺: at least 0.25 and Zn²⁺ at any of the above ratios (and optionally Fe²⁺ at no more than 1).

It is to be understood that the total metal:protein subunit ratio for any combination of Mn²⁺, Zn²⁺ and/or Fe²⁺ can be at most 2. Since not necessarily all active sites are occupied by a metal, it may however also be lower, e.g. up to 1.9, 1.7 or 1.5. In a preferred embodiment, the combined total Mn²⁺, Zn²⁺ and/or Fe²⁺:protein subunit ratio is between 1 and 2, preferably between 1.3 and 2, more preferably between 1.5 (or 1.6) and 1.9 and most preferably between 1.7 and 1.85.

In another preferred embodiment, at least 1%, at least 2% or at least 3%, preferably at least 5%, more preferably at at least 7%, and most preferably at least 10% (e.g. at least 15% or at least 19%) of the creatinine deiminase polypeptides of the plurality of the creatinine deiminase polypeptides comprises an active site to which Mn²⁺ is bound.

The isolated creatinine deiminase polypeptide may comprise a C-terminal or an N-terminal tag, preferably a His-tag.

Further, the isolated creatinine deiminase polypeptide may comprise a peptide linker between the tag and the amino acid sequence according to SEQ ID NO: 4 or the variant thereof. The length of the linker is preferably 2-20, more preferably 3-15 or 4-10 amino acids, e.g. 5 amino acids. The peptide linker preferably is cleavable, e.g. due to comprising a protease cleavage site, i.e. a cleavage site recognizable and cleavable by a protease. The protease is preferably specific to the sequence of the cleavage site, i.e. it does recognizes and cleaves the isolated creatinine deiminase polypeptide only at the cleavage site. Sequence-specific proteases are well-known. Preferably, the protease is an enteropeptidase. An exemplary cleavage site is a thrombin cleavage site (residues 4 to 9 of SEQ ID NO: 9).

In a second aspect, the present invention relates to an isolated nucleic acid encoding for a creatinine deiminase polypeptide as defined in the first aspect. This isolated nucleic acid preferably comprises a nucleotide sequence according to nucleotides 115 to 1374 of SEQ ID NO: 3 or an at least 80% sequence identity variant thereof. Nucleotides 1372 to 1374 of SEQ ID NO: 3 denote the stop codon TAA, which can be exchanged for a different stop codon TAG or TGA. It is particularly preferred, regardless of the minimum level of sequence identity, that the sequence of the variant retains one or more of nucleotides 1204, 1205, 1225, 1295, and/or 1314 of SEQ ID NO: 3.

In a preferred embodiment, the isolated nucleic acid of the second aspect comprises a Tissierella creatinini creatinine deiminase 5′ UTR. Preferably, the 5′ UTR comprises at least nucleotides 105 to 114 (range 1), at least nucleotides 95 to 114 (range 2), at least nucleotides 75 to 114 (range 3), at least nucleotides 65 to 114 (range 4), at least nucleotides 55 to 114 (range 5), at least nucleotides 45 to 114 (range 6), at least nucleotides 35 to 114 (range 7), at least nucleotides 25 to 114 (range 8), at least nucleotides 15 to 114 (range 9), at least nucleotides 5 to 114 (range 10) or at least nucleotides 1 to 114 (range 11) (with each range being preferred to the preceding one) of SEQ ID NO: 3, or an at least 80% sequence identity variant thereof. The 5′ UTR is preferably characterized in that it improves or ascertains the expression and/or the creatinine deiminase activity of the creatinine deiminase polypeptide the nucleic acid encodes. Improving or ascertaining the expression can refer to the level of total expression and/or to the amount of the expressed polypeptide in soluble form, e.g. as a proportion of the total amount including insoluble polypeptide.

Furthermore, it is preferred that the isolated nucleic acid of the second aspect comprises a Tissierella creatinini creatinine deiminase 3′ UTR, in particular in addition to the aforementioned 5′ UTR. Preferably, the 3′ UTR comprises at least nucleotides 1375 to 1394 (range 1), at least nucleotides 1375 to 1414 (range 2), at least nucleotides 1375 to 1434 (range 3), at least nucleotides 1375 to 1454 (range 4), at least nucleotides 1375 to 1474 (range 5), at least nucleotides 1375 to 1494 (range 6), at least nucleotides 1375 to 1514 (range 7), at least nucleotides 1375 to 1524 (range 8), at least nucleotides 1375 to 1544 (range 9), at least nucleotides 1375 to 1564 (range 10) or at least nucleotides 1375 to 1594 (range 11) (with each range being preferred to the preceding one) of SEQ ID NO: 3, or an at least 80% sequence identity variant thereof. It is particularly preferred, regardless of the minimum level of sequence identity, that the sequence of the variant retains nucleotide 1524 of SEQ ID NO: 3. The 3′ UTR is preferably characterized in that it improves the expression and/or the creatinine deiminase activity of the creatinine deiminase polypeptide the nucleic acid encodes. Improving expression has the meaning defined above.

In embodiments in which the isolated nucleic acid of the second aspect comprises both the 5′ and the 3′ UTR, each of the aforementioned 5′ UTR ranges can be combined with each of the aforementioned 3′ UTR ranges. It is preferred, though, that the corresponding ranges 1 are combined with each other, or the ranges 2, 3, 4, 5, 6, 7, 8, 9, 10 of 11 (with each combination being preferred to the preceding one).

In one preferred embodiment, the isolated nucleic acid of the second aspect comprises at least nucleotides 1 to 1374 of SEQ ID NO: 3 or an at least 80% sequence identity variant thereof. In a more preferred embodiment, it comprises nucleotides 1 to 1594 of SEQ ID NO: 3 or an at least 80% sequence identity variant thereof.

Optionally, for any of the above sequences, the isolated nucleic acid may comprise a nucleic acid sequence encoding for a tag as described above inserted at the 3′ or at the 5′ end of the nucleotide sequence according to nucleotides 115 to 1374 of SEQ ID NO: 3 or the variant thereof. This nucleic acid may further comprise a nucleic acid sequence encoding for a peptide linker as described above inserted between the nucleic acid sequence encoding for the tag and the nucleotide sequence according to nucleotides 115 to 1374 of SEQ ID NO: 3 or the variant thereof.

In a third aspect, the present invention relates to a vector comprising the nucleic acid of the second aspect. The vector preferably further comprises a promoter that is operatively linked to the nucleic acid of the second aspect. The promoter may be inducible or constitutive.

In a fourth aspect, the present invention relates to a cell comprising the polypeptide of the first aspect, the nucleic acid of the second aspect, or the vector of the third aspect. While the cell may be a eukaryotic cell, it preferably is a prokaryotic cell, more preferably a bacterial cell. A preferred example of a bacterial cell is an E. coli cell. The cell is not a Tissierella creatinini cell.

In a fifth aspect, the present invention relates to a method for producing an isolated creatinine deiminase polypeptide as defined in the first aspect, comprising the steps of
(i) expressing the nucleic acid as defined in the second aspect in a cell as defined in the fourth aspect, and
(ii) isolating the creatinine deiminase polypeptide.

Preferably, the isolated creatinine deiminase polypeptide is soluble.

In a preferred embodiment, the method is a method for producing a creatinine deiminase polypeptide as defined in the first aspect, wherein in step (ii) the creatinine deiminase polypeptide is isolated.

Preferably, step (i) of the method of the fifth aspect comprises culturing the cell in a medium comprising one or more metal dications. In one embodiment, the one or more metal dications are selected from the group consisting of Zn²⁺, Fe²⁺ and Mn²⁺. For example, the medium can comprise Zn²⁺ and/or Fe²⁺. It is preferred, though, that it comprises Mn²⁺, and optionally also Zn²⁺ and/or Fe²⁺. Of the optional Zn²⁺ and/or Fe²⁺, Zn²⁺ is preferred. It is envisaged that the concentration of each metal dication in the medium, in particular of Mn²⁺, is at least equimolar to the concentration of creatinine deiminase polypeptide expressed in step (i). Preferred concentrations of each metal dications in the medium, in particular of Mn²⁺, are at least 0.05 mM, preferably at least 0.1 mM, or in the range of 0.05 mM to 0.2 mM, preferably about 0.1 mM.

The medium may either comprise the one or more metal dications when added to the cell as defined in the fourth aspect, or the one or more metal dications, in particular Fe²⁺, Zn²⁺ and/or Mn²⁺, preferably Mn²⁺ (more preferably Mn²⁺ and also Zn²⁺ and/or Fe²⁺, of which Zn²⁺ is preferred), can be added to the medium in step (i), preferably when or immediately prior to inducing the expression of the nucleic acid. The medium may also comprise the one or more metal dications, in particular Fe²⁺, Zn²⁺ and/or Mn²⁺, preferably Fe²⁺ and/or Zn²⁺, when added to the cell as defined in the fourth aspect, and the medium can be further supplemented with one or more metal dications already comprised in the medium (in particular Fe²⁺, Zn²⁺ and/or Mn²⁺, preferably Fe²⁺ and/or Zn²⁺), in particular in step (i), preferably when or immediately prior to inducing the expression of the nucleic acid. Immediately prior in this respect can be up to 60 minutes, preferably up to 30 minutes and more preferably up to 5 minutes prior. In one embodiment, Mn²⁺ is added as described above to the medium comprising Fe²⁺ and/or Zn²⁺, and the medium is optionally also supplemented with Fe²⁺ and/or Zn²⁺ as described above. The inventor made the surprising finding that the addition of Mn²⁺ has a significant positive effect on producing enzymatically active creatinine deiminase polypeptide.

In a preferred embodiment, step (ii) of the method of the fifth aspect comprises lysing and centrifuging the cells and retaining the supernatant. The creatinine deiminase polypeptide is then isolated from the supernatant.

Methods for protein isolation are known in the art and include for example chromatography (including but not limited to IMAC such as Ni-chelate chromatography, and ion exchange chromatography).

It is further preferred that step (ii) of the method of the fifth aspect comprises reducing the amount of, preferably eliminating NADH-consuming enzymes. Such enzymes may be comprised in the cell of step (i).

In a sixth aspect, the present invention relates to a creatinine deiminase polypeptide produced with the method as defined in the fifth aspect. It also relates to an isolate comprising a creatinine deiminase polypeptide produced with the method as defined in the fifth aspect.

Preferably, the creatinine deiminase polypeptide is soluble.

The creatinine deiminase polypeptide produced in this manner preferably comprises an active site to which one or more metal dications selected from the group consisting of Zn²⁺, Fe²⁺, Ni²⁺ and Mn²⁺ are bound. It is preferred that at least one of the one or more metal dications is Mn²⁺. As implied by the method as defined in the fifth aspect, the isolate comprises a plurality of the creatinine deiminase polypeptides. In a particular embodiment, the molar ratio of Mn²⁺ to creatinine deiminase polypeptide in the isolate, in particular of the plurality of the creatinine deiminase polypeptides, is at least 0.05 or 0.10 (metal:protein subunit), preferably at least 0.15 (e.g. at least 0.17, 0.19, 0.21 or 0.23), and more preferably at least 0.25. The theoretical upper limit for Mn²⁺ according to the invention is 2 (a metal dication can bind to each of the two protein subunits), which can be combined with each of the afore-mentioned lower limits. In one preferred embodiment, the upper limit for Mn²⁺ is 1, which can also be combined with each of the afore-mentioned lower limits. In another preferred embodiment, the upper limit for Mn²⁺ is 0.5, which can also be combined with each of the afore-mentioned lower limits.

Preferably, in this particular embodiment, the molar ratio of Zn²⁺ to creatinine deiminase polypeptide in the isolate, in particular of the plurality of the creatinine deiminase polypeptides, is at least 0.05 or 0.15 (metal:protein subunit), preferably at least 0.25 (e.g. at least 0.3, 0.4 or 0.5), and most preferably at least 0.52. The theoretical upper limit for Zn²⁺ according to the invention is 2 minus the minimum ratio selected for Mn²⁺, e.g. 1.95 if the minimum ratio of Mn²⁺ is 0.05, or, if Fe²⁺ is also comprised (see below), it is 2 minus the minimum ratio selected for Mn²⁺ and minus the minimum ratio selected for Fe²⁺. This can be combined with each of the afore-mentioned Zn²⁺ lower limits. In one preferred embodiment, the upper limit for Zn²⁺ is 1.6, which can also be combined with each of the afore-mentioned lower limits. In another preferred embodiment, the upper limit for Zn²⁺ is 1.3, which can also be combined with each of the afore-mentioned lower limits.

Also envisaged, in this particular embodiment, is that the molar ratio of Fe²⁺ to creatinine deiminase polypeptide in the isolate, in particular of the plurality of the creatinine deiminase polypeptides, is at least 0.05 or 0.10 (metal:protein subunit), preferably at least 0.15 (e.g. at least 0.2, 0.25 or 0.3), and most preferably at least 0.42. The theoretical upper limit for Fe²⁺ according to the invention is 2 minus the minimum ratio selected for Mn²⁺, e.g. 1.95 if the minimum ratio of Mn²⁺ is 0.05, or, if Zn²⁺ is also comprised (see above), it is 2 minus the minimum ratio selected for Mn²⁺ and minus the minimum ratio selected for Zn²⁺. This can be combined with each of the afore-mentioned Fe²⁺ lower limits. In one preferred embodiment, the upper limit for Fe²⁺ is 1.2, which can also be combined with each of the afore-mentioned lower limits. In another preferred embodiment, the upper limit for Fe²⁺ is 0.7, which can also be combined with each of the afore-mentioned lower limits.

In various preferred embodiments, the molar ratios to creatinine deiminase polypeptide of the plurality of the creatinine deiminase polypeptides are as follows (metal:protein subunit; in each embodiment, the upper limit for the molar ratio is preferably 1 for Mn²⁺ and 1.5 for Zn²⁺):

Mn²⁺: at least 0.05 and Zn²⁺ at any of the above ratios (and optionally Fe²⁺ at no more than 1);
Mn²⁺: at least 0.10 and Zn²⁺ at any of the above ratios (and optionally Fe²⁺ at no more than 1);
Mn²⁺: at least 0.15 and Zn²⁺ at any of the above ratios (and optionally Fe²⁺ at no more than 1);
Mn²⁺: at least 0.17 and Zn²⁺ at any of the above ratios (and optionally Fe²⁺ at no more than 1);
Mn²⁺: at least 0.19 and Zn²⁺ at any of the above ratios (and optionally Fe²⁺ at no more than 1);
Mn²⁺: at least 0.21 and Zn²⁺ at any of the above ratios (and optionally Fe²⁺ at no more than 1);
Mn²⁺: at least 0.23 and Zn²⁺ at any of the above ratios (and optionally Fe²⁺ at no more than 1);
Mn²⁺: at least 0.25 and Zn²⁺ at any of the above ratios (and optionally Fe²⁺ at no more than 1).

It is to be understood that the total metal:protein subunit ratio for any combination of Mn²⁺, Zn²⁺ and/or Fe²⁺ can be at most 2. Since not necessarily all active sites are occupied by a metal, it may however also be lower, e.g. up to 1.9, 1.7 or 1.5. In a preferred embodiment, the combined total Mn²⁺, Zn²⁺ and/or Fe²⁺:protein subunit ratio is between 1 and 2, preferably between 1.3 and 2, more preferably between 1.5 (or 1.6) and 1.9 and most preferably between 1.7 and 1.85. It is also to be understood that, when it is referred to the isolate, the isolate may comprise further metal ions not bound to the creatinine deiminase polypeptides if these are not removed during isolation.

In another preferred embodiment, at least 1%, at least 2% or at least 3%, preferably at least 5%, more preferably at at least 7%, and most preferably at least 10% (e.g. at least 15% or at least 19%) of the creatinine deiminase polypeptides of the plurality of the creatinine deiminase polypeptides comprises an active site to which Mn²⁺ is bound.

In a seventh aspect, the present invention relates to the use of a creatinine deiminase polypeptide as defined in the first or sixth aspect or of the isolate of the sixth aspect for determining the amount of creatinine in a sample. In particular, it relates to a method for determining the amount of creatinine in a sample, comprising the steps of
(a) contacting the sample with the creatinine deiminase polypeptide as defined in the first or sixth aspect or with the isolate as defined in the sixth aspect to convert the creatinine in the sample to N-methylhydantoin and NH_{3/4}⁺, and
(b) quantifying the conversion of step (a).

The quantity of the conversion, e.g. the decrease of precursors of the conversion or the increase of products of the conversion, indicates the amount of creatinine. For this purpose, values of a reference conversion with a known creatinine amount can be used.

Upon the contacting of step (a), NH_{3/4}⁺ is produced. NH_{3/4}⁺ herein means NH₃ or NH₄⁺, or a mixture of both compounds (i.e. both NH₃ or NH₄⁺ are produced). In an aqueous solution, NH₃ and NH₄⁺ are present in an equilibrium. The balance of the equilibrium is pH-dependent, the pKₐ value for NH₃ is 9.25). At pH values of less than 9.25, the conversion produces more NH₄⁺ than NH₃, and at pH values of more than 9.25, the conversion produces more NH₃ than NH₄⁺. Substantially the same amount of NH₃ and NH₄⁺ is produced at a pH of 9.25.

The sample is usually, but not necessarily a sample from a subject, preferably a body fluid sample. Preferred body fluid samples are blood, serum, plasma and urine.

The subject is preferably is selected from the group consisting of laboratory animals (e.g. mouse or rat), domestic animals (including e.g. guinea pig, rabbit, horse, donkey, cow, sheep, goat, pig, chicken, camel, cat, dog, turtle, tortoise, snake, or lizard), or primates including chimpanzees, bonobos, gorillas, and humans. Humans are particularly preferred.

In a preferred embodiment, step (b) of the method comprises determining the amount of NH_{3/4}⁺ or of N-methylhydantoin produced in step (a). This can be achieved, for example, in an embodiment wherein step (a) further comprises contacting the sample with NADPH (or NADH), α-ketoglutarat and glutamatdehydrogenase to convert NH_{3/4}⁺ (preferably NH₄⁺), α-ketoglutarat and NADPH (or NADH) to glutamate and NADP⁺ (or NAD⁺, respectively), and step (b) comprises quantifying the consumption of NADPH (or NADH, respectively). This further contacting can occur prior to, simultaneous to or after the contacting of the sample with the creatinine deiminase polypeptide or the isolate. Suitable assays are known in the art, e.g. from Tanganelli, Clin. Chem. 28/7, 1461-1484 (1982). Preferably, the concentration of added NADPH (or NADH) is from 0.01 to 0.2 mM, more preferably from 0.05 to 0.15 mM, most preferably 0.01 mM. The inventor found that in this range, the linear range of consumption is broader. Also, in addition or independent from this NADPH (or NADH) concentration, the concentration of the added creatinine deiminase polypeptide is 10 mg/ml or less, 5 mg/ml or less, 1 mg/ml or less, preferably from 0.05 to 0.01 mg/ml (or about 0.02 mg/ml), more preferably from 0.01 to 0.003 mg/ml (or about 0.005 mg/ml), or most preferably from 0.003 to 0.001 mg/ml (or about 0.002 mg/ml). The inventor found that at these concentrations, the enzyme activity is particularly advantageous.

In a particular preferred embodiment, the creatinine deiminase polypeptide is the last substance to be contacted with the sample in step (a). In other words, the addition of the creatinine deiminase polypeptide to the conversion reaction starts the conversion.

The consumption of NADPH (or NADH) can be determined optically, more specifically photometrically or fluorimetrically, for example. Photometric determination includes measuring disappearance or the rate of disappearance of NADPH (or NADH) light absorption of the reaction mix, preferably at or near 340 nm (e.g. +/−15 nm), see e.g. Tanganelli, Clin. Chem. 28/7, 1461-1484 (1982). Fluorimetric determination includes measuring disappearance or the rate of disappearance of NADPH (or NADH) fluorescence, preferably at or near 460 nm (e.g. +/−15 nm), with an excitation wavelength preferably at or near 340 nm (e.g. +/−15 nm), see e.g. Chen at al., Clin. Chem. Acta, 100 (1980) 21.

The production of NH_{3/4}⁺ can be determined with optical sensors, e.g. with colorimetric dry slides. Suitable colorimetric dry slides are known in the art, see e.g. Tofaletti et al., Clin. Chem. 29/4, 684-687, 1983. In such sensors, the creatinine deiminase polypeptide is embedded in a dry film. The NH_{3/4}⁺ produced by the conversion catalysed by the creatinine deiminase polypeptide in the sample diffuses through a membrane, preferably a semipermeable membrane, and is measured by the color generated by its reaction with a pH-sensitive dye. The term “semipermeable membrane” refers to a membrane that is permeable for NH_{3/4}⁺, but not for the creatinine deiminase polypeptide. This is achieved by a suitable pore size of the membrane. Suitable membranes are known in the art, e.g. from Tofaletti et al., Clin. Chem. 29/4, 684-687, 1983. In preferred embodiments, the pore size is less than 10 nm, preferably less than 1 nm.

The conversion started in step (a) can also be determined electrochemically, for example by one or more electrodes measuring in step (b) products of the conversion, including for example hydrogen ions (e.g. the change of pH) or ammonium ions. Preferably, the creatinine deiminase polypeptide is immobilized directly on the surface of the one or more electrode, e.g. in form of a layer (e.g. as a layer or within a layer). Suitable electrodes can also be described as sensors or biosensors as in the art, see e.g. Guilbault and Coulet, Anal. Letters 13(B18) 1607-1624; Guilbault and Coulet, Anal. Chim. Acta, 152, 223-228, 1983; Cou et al., IEEE Sensors J, 9, 665-672, 2009; Zinchenko et al., Biosens Bioelectr 35, 466-469, 2012.

In an eighth aspect, the present invention relates to a reagent kit (or simply kit) suitable for determining the amount of creatinine in a sample, preferably as defined in the seventh aspect, comprising the creatinine deiminase polypeptide as defined in the first or the sixth aspect, or the isolate as defined in the sixth aspect. In a preferred embodiment, the kit further comprises in separate containers one or more components selected from the group consisting of a buffer (preferably a pH-buffer) suitable for the use or method of the seventh aspect, a second enzyme and/or a substrate for it (e.g. glutamatdehydrogenase and/or α-ketoglutarat), and NADPH (or NADH). In a preferred embodiment, the reagent kit comprises glutamatdehydrogenase, α-ketoglutarat and one of NADPH or NADH. Preferably, it further comprises the buffer.

In a ninth aspect, the present invention relates to a composition, preferably a sensor composition suitable for determining the amount of creatinine in a sample, preferably as defined in the seventh aspect, comprising the creatinine deiminase polypeptide as defined in the first or the sixth aspect. The composition further comprises a sensor, preferably an electrode or an optical sensor (including a dry slide, preferably a colorimetric dry slide) on which the creatinine deiminase polypeptide is immobilized. In other words, when the composition is described as a sensor composition, the sensor preferably is an electrode or an optical sensor (including a dry slide, preferably a colorimetric dry slide) on which the creatinine deiminase polypeptide is immobilized. It is preferred that the composition is an electrode composition, i.e. it comprises, preferably consists of the electrode on which the creatinine deiminase polypeptide is immobilized. The term sensor refers to a device that can detect and preferably quantify one or more products of the conversion(s) started in step (a) of the method of the seventh aspect. It is intended to include, without limitation, biosensors, chemical sensors and electrical sensors. “Immobilized” herein refers to an immobilisation on (e.g. on the surface of) or in the sensor, preferably in form of a layer, e.g. as a layer or within a layer. “As a layer” means the creatinine deiminase polypeptide makes up the layer, and “within a layer means a further substance makes up the layer, and the creatinine deiminase polypeptide the layer is embedded within that layer.

In a tenth aspect, the present invention relates to an in vitro method for detecting, preferably diagnosing kidney disease in a subject, comprising determining the amount of creatinine in a sample from the subject as defined in the seventh aspect, wherein an amount of creatinine that is larger than the normal value indicates that the subject has kidney disease. A normal value can be known in the art or determined from one or more control subject samples. A control subject is a subject not having kidney disease. Normal values are known in the art, and exemplary normal values are 58-110 μmol/L for males and 46-92 μmol/L for females, both in serum, or 8840-17680 μmol/day for males and 7072-15912 μmol/day for females in urine. The normal value is preferably adjusted for one or more of age, race, gender and body weight of the subject.

In one embodiment, the kidney disease is characterized by a decrease in nephron function. In a preferred embodiment, the kidney disease is stage III kidney disease (glomerular filtration rate, GFR of 30-59), stage IV kidney disease (GFR of 15-29) or stage V kidney disease (GFR below 15).

In a particularly preferred embodiment, the method further comprises determining the amount of albumin in the sample, wherein an albumin-to-creatinine ratio (ACR) of 30 or higher, preferably of 300 of higher indicates that the subject has kidney disease.

The method of the tenth aspect may further comprise a step of treating the kidney disease. Also envisaged is a method of treating kidney disease, wherein the subject has been diagnosed according to the method of the tenth aspect. Treating may include, for example, administering one or more medicaments selected from the group consisting of a medicament reducing blood pressure, a medicament reducing the cholesterol level, a medicament treating anemia, and a medication relieving swelling. Treating may also include dialysis and/or a kidney transplant.

Definitions and embodiments described below, in particular under the header ‘Definitions and further embodiments of the invention’ apply to all of the above-described aspects of the invention. Also, definitions given and embodiments described with respect to any of the above-described aspects apply also to all other aspects, in as far as they are applicable.

### Definitions and Further Embodiments of the Invention

The specification uses a variety of terms and phrases, which have certain meanings as defined below. Preferred meanings are to be construed as preferred embodiments of the aspects of the invention described herein. As such, they and also further embodiments described in the following can be combined with any embodiment of the aspects of the invention and in particular any preferred embodiment of the aspects of the invention described above.

As used herein, the term “isolated” refers to a molecule which is substantially free of other molecules with which it is naturally associated with. In particular, isolated means the molecule is not in an animal body or an animal body sample. An isolated molecule is thus free of other molecules that it would encounter or contact in an animal. Isolated does not mean isolated from other components associated with as described herein, e.g. not isolated from other components of a composition the molecule is comprised in, or isolated from a vector or cell it is comprised in.

As used herein, the term “creatinine deiminase” (EC number 3.5.4.21), also known as creatinine deaminase, creatinine desaminase or desiminase, creatinine iminohydrolase or creatinine hydrolase, refers to an enzyme catalysing the reaction of creatinine to L-methylhydantoin and NH_{3/4}⁺.

The term “variant” refers, with respect to a polypeptide, generally to a modified version of the polypeptide, e.g. a mutation, so one or more amino acids of the polypeptide may be deleted, inserted, modified and/or substituted. More specific functions are defined herein and have precedence over the general definition. A “mutation” or “amino acid mutation” can be an amino acid substitution, deletion and/or insertion (“and” may apply if there is more than one mutation). Preferably, it is a substitution (i.e. a conservative or non-conservative amino acid substitution), more preferably a conservative amino acid substitution. In some embodiments, a substitution also includes the exchange of a naturally occurring amino acid with a not naturally occurring amino acid. A conservative substitution comprises the substitution of an amino acid with another amino acid having a chemical property similar to the amino acid that is substituted. Preferably, the conservative substitution is a substitution selected from the group consisting of:
(i) a substitution of a basic amino acid with another, different basic amino acid;
(ii) a substitution of an acidic amino acid with another, different acidic amino acid;
(iii) a substitution of an aromatic amino acid with another, different aromatic amino acid;
(iv) a substitution of a non-polar, aliphatic amino acid with another, different non-polar, aliphatic amino acid; and
(v) a substitution of a polar, uncharged amino acid with another, different polar, uncharged amino acid.

A basic amino acid is preferably selected from the group consisting of arginine, histidine, and lysine. An acidic amino acid is preferably aspartate or glutamate. An aromatic amino acid is preferably selected from the group consisting of phenylalanine, tyrosine and tryptophane. A non-polar, aliphatic amino acid is preferably selected from the group consisting of glycine, alanine, valine, leucine, methionine and isoleucine. A polar, uncharged amino acid is preferably selected from the group consisting of serine, threonine, cysteine, proline, asparagine and glutamine. In contrast to a conservative amino acid substitution, a non-conservative amino acid substitution is the exchange of one amino acid with any amino acid that does not fall under the above-outlined conservative substitutions (i) through (v).

Means for determining sequence identity are described below.

Amino acids of a protein may also be modified, e.g. chemically modified. For example, the side chain or a free amino or carboxy-terminus of an amino acid of the protein or polypeptide may be modified by e.g. glycosylation, amidation, phosphorylation, ubiquitination, etc. The chemical modification can also take place in vivo, e.g. in a host-cell, as is well known in the art. For example, a suitable chemical modification motif, e.g. glycosylation sequence motif present in the amino acid sequence of the protein will cause the protein to be glycosylated. Unless a modification leads to a change in identity of a modified amino acid (e.g. a substitution or deletion), a modified polypeptide is within the scope of polypeptide as mentioned with respect to a certain SEQ ID NO, i.e. it is not a variant as defined herein.

The term “variant” refers, with respect to a polynucleotide, generally to a modified version of the polynucleotide, e.g. a mutation, so one or more nucleotides of the polynucleotide may be deleted, inserted, modified and/or substituted. More specific functions are defined herein and have precedence over the general definition. A “mutation” can be a nucleotide substitution, deletion and/or insertion (“and” may apply if there is more than one mutation). Preferably, it is a substitution, more preferably it causes an amino acid substitution, most preferably a conservative amino acid substitution.

The term “identity” or “identical” in the context of polynucleotide, polypeptide or protein sequences refers to the number of residues in the two sequences that are identical when aligned for maximum correspondence. Specifically, the percent sequence identity of two sequences, whether nucleic acid or amino acid sequences, is the number of exact matches between two aligned sequences divided by the length of the shorter sequence and multiplied by 100. Alignment tools that can be used to align two sequences are well known to the person skilled in the art and can, for example, be obtained on the World Wide Web, e.g., Clustal Omega (http://www.ebi.ac.uk/Tools/msa/clustalo/) for polypeptide alignments or MUSCLE (http://www.ebi.ac.uk/Tools/msa/muscle/) or MAFFT (http://www.ebi.ac.uk/Tools/msa/mafft/) for polynucleotide alignments or WATER (http://www.ebi.ac.uk/Tools/psa/emboss_water/) for polynucleotide and polypeptide alignments. The alignments between two sequences may be carried out using default parameters settings, e.g. for MAFFT preferably: Matrix: Blosum62, Gap Open 1.53, Gap Extend 0.123, for WATER polynucleotides preferably: MATRIX: DNAFULL, Gap Open: 10.0, Gap Extend 0.5 and for WATER polypeptides preferably MATRIX: BLOSUM62, Gap Open: 10.0, Gap Extend: 0.5. Those skilled in the art understand that it may be necessary to introduce gaps in either sequence to produce a satisfactory alignment. The “best sequence alignment” is defined as the alignment that produces the largest number of aligned identical residues while having a minimal number of gaps. Preferably, it is a global alignment, which includes every residue in every sequence in the alignment.

The term “polynucleotide” is intended to refer to a nucleic acid, i.e. a biological molecule made up of a plurality of nucleotides. It includes DNA, RNA and synthetic analogs, e.g. PNA. DNA is preferred.

The description above refers generally to “at least 80% sequence identity variants”. In preferred embodiments thereof, the variant is an at least 83%, at least 85% or at least 90%, more preferably an at least 95%, 96%, 97%, 98% or most preferably an at least 99% sequence identity variant, all with respect to the respective SEQ ID NO or part thereof referred to.

The term “creatinine deiminase activity” refers to the ability of an enzyme to catalyse the reaction creatinine+H₂O [symbol] N-methylhydantoin+NH₃. The activity is usually expressed in U/ml and can be determined in volumetric activity assays as e.g. in the examples according to the following equation:

$$U \text{/} ml = \frac{ΔE * {min}^{- 1} * {V}_{total} * dil}{ɛ * {V}_{sample} * d}$$

wherein:
ΔE: Optical density difference
ε: specific absorption coefficient for NADH at the used wavelength (6.22 ml μmol-¹ cm-¹)
dil: dilution
d: path length of beam in cuvette
Vₜₒₜₐₗ: total volume
Vₛₐₘₚₗₑ: sample volume

The term “soluble”, as used herein, unless otherwise specified, refers to those proteins having a protein solubility in an aqueous solution (e.g. water) of at least about 40%, including from 50% to 100%, and also including from 60% to 90%, preferably 90% to 100%, for examples as measured in accordance with the following process: (1) suspend protein in purified water at 5.00 g per 100 g of suspension; (2) adjust the pH of the suspension to a desired pH (e.g. 7); (3) stir gently at room temperature (e.g. 20° C.-22° C.) for 60 minutes; (4) measure total protein in the suspension by any suitable technique (e.g. HPLC); (5) centrifuge an aliquot of the suspension e.g. at 16,000×g and at 4° C. for 30 minutes; (6) measure the supernatant for protein by the selected technique as described in step (4); and (7) calculate protein solubility as the supernatant protein percentage of the total protein. In a preferred embodiment, it means that least about 40%, including from 50% to 100%, and also including from 60% to 90%, preferably 90% to 100% of the protein expressed in E. coli are contained in the aqueous supernatant rather than the pellet after centrifugation at 16,000 g at 4° C. for 30 minutes of an E. coli cell lysate.

The term “UTR” (“untranslated region”) refers to a nucleotide sequence of an mRNA or of DNA that is transcribed into mRNA, which is not translated into a polypeptide sequence.

The term “ascertains the creatinine deiminase activity” refers to a feature being required for creatinine deiminase activity. The term “improves the creatinine deiminase activity” refers to a feature increasing the creatinine deiminase activity, e.g. by at least 10%, by at least 25%, by at least 50%, or by at least 100%. Methods for testing creatinine deiminase activity, including whether a feature ascertains or improves creatinine deiminase activity, are disclosed herein.

The term “tag” refers to a heterologous polypeptide sequence that is recombinantly attached to a polypeptide. Preferred is a tag suitable for allowing for purification and/or quantification. Tags may e.g. encompass affinity tags, chromatography tags, epitope tags, or fluorescence tags. Affinity tags are appended to proteins so that they can be purified from a biological source using an affinity technique. These include chitin binding protein (CBP), maltose binding protein (MBP), and glutathione-S-transferase (GST). The poly(His) tag is a widely used protein tag which binds to metal matrices. Chromatography tags are used to alter chromatographic properties of the protein to afford different resolution across a particular separation technique. Often, these consist of polyanionic amino acids, such as FLAG-tag. Epitope tags are short peptide sequences which are chosen because high-affinity antibodies can be reliably produced in many different species. These are usually derived from viral genes, which explain their high immunoreactivity. Epitope tags include V5-tag, Myc-tag, and HA-tag. These tags are particularly useful for western blotting, immunofluorescence and immunoprecipitation experiments, although they also find use in antibody purification. Fluorescence tags are used to give visual readout on a protein. GFP and its variants (e.g. mutant GFPs having a different fluorescent spectrum) and RFP and its variants (e.g. mutant RFPs having a different fluorescent spectrum) are the most commonly used fluorescence tags. More advanced applications of GFP/RFP include using it as a folding reporter (fluorescent if folded, colorless if not). Further examples of fluorophores include fluorescein, rhodamine, and sulfoindocyanine dye Cy5. Preferred examples of a tag include but are not limited to AviTag, Calmodulin-tag, polyglutamate tag, E-tag, FLAG-tag, HA-tag, His-tag (preferably 5-10, e.g. 6 histidines bound by a nickel or cobalt chelate), Myc-tag, S-tag, SBP-tag, Softag 1, Softag 3, Strep-tag, TC tag, V5 tag, VSV-tag, Xpress tag, Isopeptag, SpyTag, BCCP, Glutathione-S-transferase-tag, Green fluorescent protein-tag, Maltose binding protein-tag, Nus-tag, Thioredoxin-tag, Fc-tag, or Ty tag. Preferred is a His-tag.

As used herein, the term “vector” refers to a protein or a nucleic acid or a mixture thereof which is capable of being introduced or of introducing a polynucleotide comprised therein into a cell, and optionally expressing the polynucleotide in the cell. In the context of the present invention it is preferred that the nucleic acid of the second aspect is expressed within the cell upon introduction of the vector. Suitable vectors are known in the art and include, for example, plasmids, cosmids, artificial chromosomes (e.g. bacterial, yeast or human), bacteriophages, viral vectors (e.g. retroviruses, lentiviruses, adenoviruses, adeno-associated viruses or baculoviruses), or nano-engineered substances (e.g. ormosils). Required vector technologies are well known in the art (see e.g. Lodish et al., Molecular Cell Biology, W. H. Freeman; 6th edition, Jun. 15, 2007; or Green and Sambrook, Molecular Cloning—A Laboratory Manual, 2012 Cold Spring Harbor Laboratory Press). The term includes cloning vectors and in particular expression vectors.

The term “promoter” as used herein refers to a sequence of DNA that directs the transcription of a gene. A promoter may be “inducible”, initiating transcription in response to a promoter activating agent, or it may be “constitutive”, whereby the regulation of the transcription is independent of such an agent. Preferred are promoters of bacterial or viral origin. Suitable promoters are known in the art.

The term “operatively linked” as used herein refers to elements or structures in a nucleic acid sequence that are linked by operative ability and not physical location. The elements or structures are capable of, or characterized by accomplishing a desired operation. It is recognized by one of ordinary skill in the art that it is not necessary for elements or structures in a nucleic acid sequence to be in a tandem or adjacent order to be operatively linked.

The cell referred to above may be any prokaryotic or eukaryotic cell. The prokaryotic cell can be any kind of bacterial or archeal organism suitable for application in recombinant DNA technology such as cloning or protein expression, including both Gram-negative and Gram-positive microorganisms. Suitable bacteria may be selected from e.g. Escherichia (in particular E. coli, which is most preferred), Anabaena, Caulobacter, Gluconobacter, Rhodobacter, Pseudomonas, Paracoccus, Bacillus, Brevibacterium, Cupriavidus, Corynebacterium, Rhizobium (Sinorhizobium), Flavobacterium, Klebsiella, Enterobacter, Lactobacillus, Lactococcus, Methylobacterium, Propionibacterium, Staphylococcus or Streptomyces.

A eukaryotic cell is in particular a fungal or an animal cell. A fungal cell can be, in the broadest sense, any cell of a fungal organism, for example a cell from Kluyveromyces lactis, Kluyveromyces marxianus var. marxianus, Kluyveromyces thermotolerans, Candida utilis, Candida tropicalis, Candida albicans, Candida lipolytica and Candida versatilis, of the genus Pichia like Pichia stipidis, Pichia pastoris and Pichia sorbitophila, Cryptococcus, Debaromyces, Hansenula, Saccharomycecopsis, Saccharomycodes, Schizosaccharomyces, Wickerhamia, Debayomyces, Hanseniaspora, Kloeckera, Zygosaccharomyces, Ogataea, Kuraishia, Komagataella, Metschnikowia, Williopsis, Nakazawaea, Cryptococcus, Torulaspora, Bullera, Rhodotorula, Willopsis or Sporobolomyces. Preferably, though, the fungal cell is a Saccharomyces or Pichia cell, in particular a Saccharomyces cerevisiae or a Pichia pastoris cell.

An animal cell may be a cell of a primate, mouse, rat, rabbit, dog, cat, hamster, cow, insect (e.g. Sf9 or Sf21) etc., preferably a human.

The term “cell culture” refers to the process by which cells are grown under controlled conditions outside of their natural environment in or on a cell culture medium. The term “cell culture medium” refers to a liquid or gel for supporting the survival or growth of cells, especially cells as defined above. Such a medium comprises all nutrients required to support the survival or growth of such cells. The cell culture medium composition can be a dry powder composition, a liquid composition or a solid (e.g. gel or agar) composition. Suitable cell cultures and culturing techniques are well known in the art, see for example Peterson et al., Comp Immunol Microbiol Infect Dis. 1988; 11(2):93-8.

The expression “added to the medium” with respect to the addition of a metal dication to a medium includes embodiments in which the same metal dication may or may not be already comprised in the medium. Preferably, it is not already comprised in the medium. The expression “supplemented” with respect to the addition of a metal dication to a medium means that the amount of the metal dication (that is already comprised in the medium) in the medium, is increased. “Not already comprised” in this respect includes trace amounts, and “comprised” means more than a trace amount. As used herein, the term “trace amount” refers to an amount in the level of femtomolar (fM) or less. Specifically, the “trace amount” may refer to an amount of 500 fM or less, more specifically 100 fM or less, most specifically 50 fM or less.

The expression “amount of creatinine in a sample” includes the absolute amount, e.g. the amount in mol or the mass in grams, and the relative amount (i.e. concentration), e.g. the amount in mol or the mass in grams per volume.

The term “converting” refers to a chemical conversion of one or more reagents by means of an enzymatic reaction.

The expression “substantially the same amount of NH₃ or NH₄⁺” refers to one of NH₃ or NH₄⁺ being present in the same amount ±10% (preferably ±5%, more preferably ±1%) as the other.

The term “NAD(P)H” means “NADPH or NADH”, and the term “NAD(P)⁺” means “NADP⁺ or NAD⁺”.

The term “dry slide” refers to a layered, coated dry film, which is hydraded by adding an aqueous fluid (such as a sample as described herein). The coating is preferably a creatinine deiminase (as described herein) coating.

### SEQ IDs Referred to in the Application

The present application refers to SEQ ID NOs 1-4. An overview of these SEQ IDs is given in the following:

SEQ ID NO: 1 represents the amino acid sequence of the creatinine deiminase of EP 1 325 958 A1.

SEQ ID NO: 2 represents the nucleic acid sequence of the creatinine deiminase of EP 1 325 958 A1.

SEQ ID NO: 3 represents the nucleic acid sequence of the creatinine deiminase of the invention (start and stop codons of the coding region are underlined, and nucleotides differing from SEQ ID NO: 1 are marked in bold letters):

CTGGCATTAGTGTTATTGGCTATAGCAACAATTTTGTCAATAACTGATAAA
AATACATTAACAAAAGAAAAACTGTAAGCTATTAACAATGCTAAATTTTTA
AGGAGTGATTTTATGATGAAAAAGTTTATTAATGCAAAGATTTACAAGAAC
AATGAAGCAACAGAAATTTTAGTAGAAGACGGTAAAATCAAAGAGATTGGT
AATAACTTAGCAGACTGTAAAGAAGTAATTGATCTAGGCGGTAAAATGGTT
ACTCCACCTTATGTAGATCCTCACCTACATTTAGATTATGTGTATACATTG
GCTGAACTTGGAAAAACTGGTGCTGGCTCAGGAACTCTTTTTGAAGCTATT
GAAATGTGGCCAGTATTTAAAAAGACTTTAACTGTAGAAAGCGTTAAAAAA
CTTGCTCTTAAGGGGGTTATGGATGAGGTTTCCCAAGGGGTACAACATATT
CGTACACATATAGATGTAACTGATCCAAAATTCACAGGTCTAAAAGCTATG
TTGGAAATGAAAGAAGAATTAAAGGACATAGTTGATATCCAAATAGTATCA
TTCCCACAACAAGGAATGTACACATATAAGGGTGGACGTGAATTAGTAGAA
GAAGCACTTAAGATGGGTGCAGATGTTGTTGGAGGAATTCCGCATTATGAA
CCAGCTAGAGAATATGGTGAAATGTCTGTTAAAGCCACAGTTGAACTTGCT
ATGAAATATGATAAGCTAATAGATGTTCACTGTGATGAGACAGATGATCCT
CAAGCACGTTTTATTGAGCTATTAAATGCACTTGTTTATTTGGAAGGTTAT
GGTGCAAAAACTTCAGCTAGCCATACTTGTTCATTTGGTTCAGCAGATGAT
TCATATGCATATAGAATGATAGACTTATTCAAAAAGAGCAAGATAAACTTC
ATCTCTAATCCAACTGAAAATGCGTATCTACAAGGCCGTCATGACACTTAT
CCAAAGCGTCGTGGATTGACTAGAGTTAAAGAATTTATGGAGCATGGTATT
AATGTTGCATTTGCACAAGATTCAATAAACGATCCATGGTATCCAATGGGT
AACGGAAATATGATGAATATACTTGACAATGGAATTCATTTAGCTCAAATA
ATGTCACCACAAGATATAGAAAAAGATTTAGATTTAATTACCTACAATGGT
GCTCGTTGCCTAAATATCCAAGATAAATATGGATTAGAAGTAGGTAAAGAT
GCAAACTTTATCGTTCTTAACGGAGACAGCCCATTCGATGTAATAAGAAAC
CGTGCTAATGTTCTTGCTTCTGTTAGAAAAGGAGAATTCCTATTTAAGCAA
AAACCAGTAGAATATGATGTAAAACTTGATTTAGGCGTAAAATATTAATAT
TTTAAAATAAATTCCAAATTAACCCCCCGGTGGTGTAATAAACTCCATCGG
GGGGTTTTTTGTGTTCCAGTAGAAAATAAAAAAATGATATAAAAATTTAGT
AGTTTGAAAAACTTAAATAAAGAAAGGGCGGATTTAGAATGAGTCAAAGAG
ACGTATTATATTCACCAGATGCAAAGTACAAAGATAATAAGGGTAAATATG
GAATTGATTTAGG

SEQ ID NO: 4 represents the amino acid sequence of the creatinine deiminase of the invention (amino acids differing from SEQ ID NO: 2 are marked in bold letters):

MMKKFINAKIYKNNEATEILVEDGKIKEIGNNLADCKEVIDLGGKMVTPPY
VDPHLHLDYVYTLAELGKTGAGSGTLFEAIEMWPVFKKTLTVESVKKLALK
GVMDEVSQGVQHIRTHIDVTDPKFTGLKAMLEMKEELKDIVDIQIVSFPQQ
GMYTYKGGRELVEEALKMGADVVGGIPHYEPAREYGEMSVKATVELAMKYD
KLIDVHCDETDDPQARFIELLNALVYLEGYGAKTSASHTCSFGSADDSYAY
RMIDLFKKSKINFISNPTENAYLQGRHDTYPKRRGLTRVKEFMEHGINVAF
AQDSINDPWYPMGNGNMMNILDNGIHLAQIMSPQDIEKDLDLITYNGARCL
NIQDKYGLEVGKDANFIVLNGDSPFDVIRNRANVLASVRKGEFLFKQKPVE
YDVKLDLGVKY

SEQ ID NO: 5 represents the amino acid sequence of an exemplary linker. SEQ ID NO: 6 represents an exemplary nucleotide sequence for the linker according to SEQ ID NO: 5. SEQ ID NO: 7 represents an exemplary amino acid sequence including a thrombin cleavage site. SEQ ID NO: 8 represents an exemplary nucleotide sequence for the amino acid sequence according to SEQ ID NO: 7. See the legend of Table 1 for further details.

The invention is described by way of the following examples which are to be construed as merely illustrative and not limitative of the scope of the invention.

### Example 1: Isolation of a DNA Fragment Encoding Creatinine Deiminase from Genomic DNA of Tissierella creatinini

Genomic DNA isolated from the strain Tissierella creatinini deposited at the DSMZ strain collection [https://www.dsmz.de/] under the number DSM 9508 (type strain) was purchased from DSMZ. This DNA was used as template to amplify a corresponding DNA fragment as described in EP 1 325 958 A1 using forward and reverse primers having the identical sequences of the respective ends of the published DNA sequence (SEQ ID NO: 2) and containing in addition a HindIII restriction site at the 5′ ends. Q5^{R} High-Fidelity DNA Polymerase and the respective buffer were purchased from New England BioLabs. The PCR conditions were as following:

| | |
|---|---|
| Genomic DNA (25 ng/μL) | 1 μL |
| Primer forward (10 μM) | 2.5 μL |
| Primer reverse (10 μM) | 2.5 μL |
| dNTP mix (10 mM each) | 1 μL |
| Buffer (5 × Q5 Reaction Buffer) | 10 μL |
| Polymerase Q5 | 0.2 μL |
| Aqua dest | 32.8 μL |

The temperature program parameters were denaturation at 98° C. for 10 min, 35 cycles (98° C. for 30 sec, 55° C. for 30 sec, 72° C. for 2 min) and final extension at 72° C. for 4 min.

DNA fragments of the expected size were obtained as analyzed by agarose gel electrophoresis. The fragments of 5 reactions were extracted from agarose gel using a gel extraction kit (GeneJET Gel Extraction Kit, ThermoFisher). The resulting DNA was ligated into the plasmid pBluescript II KS+, and resulting clones were analyzed by restriction analysis. The insert of one selected proper clone was sequenced. The resulting DNA sequence is shown in SEQ ID NO: 3. Comparison and analysis of the sequences of SEQ ID NO: 2 and in SEQ ID NO: 3 revealed that the genomic DNA, besides 4 base substitutions, had an additional base (C, nucleotide position 1314 in SEQ ID NO: 3) within the coding region. This results in significant differences in the amino acid sequences of the C-terminal part as indicated in SEQ ID NO: 4 (compared to SEQ ID NO: 1, the amino acid sequence of the EP 1 325 958 A1 creatinine deiminase).

### Example 2: Recombinant Expression of Creatinine Deiminase—Basic Constructs

A synthetic DNA fragment as defined in SEQ ID NO: 2 was purchased (Life Technologies—Thermo Fisher Inc.). This fragment consisted of the coding region of creatinine deiminase and the 5′ upstream and 3′ downstream regions as described in EP 1 325 958 A1. For cloning into the vector pBluescript II KS+, restriction endonuclease recognition sequences for HindIII restriction endonuclease were added at the 3′ and 5′ ends (underlined in the depiction of SEQ ID NO: 2 above). Cloning, cultivation, DNA and protein analysis was performed according to standard methods as described in Current Protocols in Molecular Biology, Wiley, Print ISSN: 1934-3639. In strategy 1 the entire fragment including the 5′ and 3′ noncoding regions was cloned via HindIII into pBluescript II KS+. The Escherichia coli strain XL1 (Stratagene) was used for all cloning and expression work as host organism. Following restriction analysis of a set of 10 transformants isolated from selective LB-Agar plates containing 100 mg/L Ampicillin, one clone of the orientation shown in FIG. 1 was selected (also referred to orientation 1 herein). For control purposes clones of the opposite orientation were examined for expression ability (also referred to orientation 2 herein). The same cloning strategy was used to clone the corresponding fragment derived from the genomic DNA of T. creatinini as defined in SEQ ID NO: 3 into pBluescript II KS+. A further clone was constructed according to strategy 1. Here the 3′ non coding region was replaced by a His tag introduced via PCR using a respective primer containing a His6 coding region fused to the C-terminus of the creatinine deiminase (long version, see schematic in Example 3, clone 4h).

In addition, following strategy 2, only the coding region was cloned into the tac-promoter based E. coli expression vector pMS470Δ8, and in two further versions as N-terminally and C-terminally His tagged (His6) protein. Therefore, the coding region was PCR amplified under standard conditions using primers fitting to the N-terminal or C-terminal parts of the coding region and containing the 6 His codons. As template the synthetic or genomic DNA fragment according to SEQ ID NO: 2 or SEQ ID NO: 3, respectively, was used.

From all strategies one proper clone was selected based on restriction analysis and the correctness of all expression clones was verified by sequencing. A schematic of the resulting constructs obtained by these cloning strategies is shown in FIG. 1.

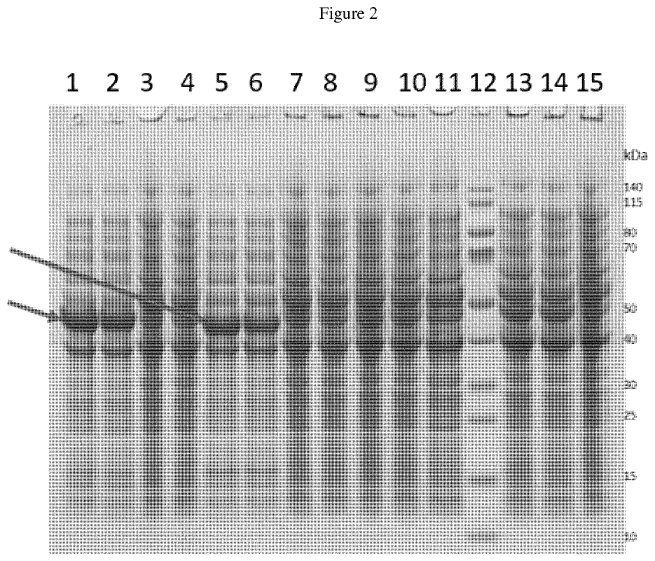

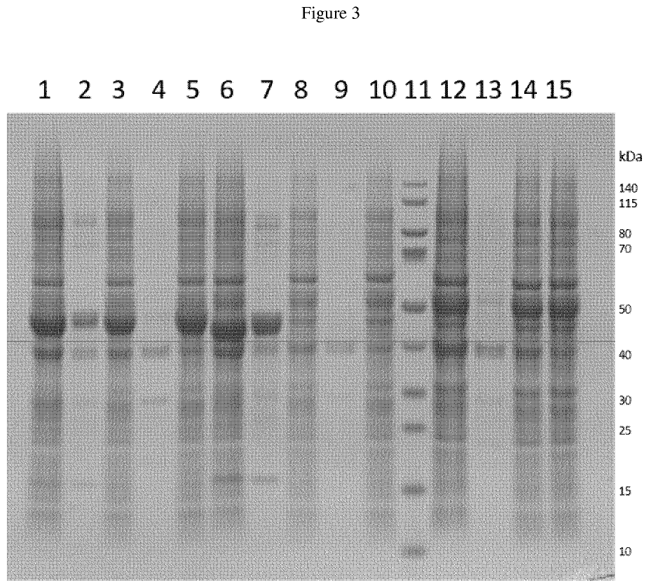

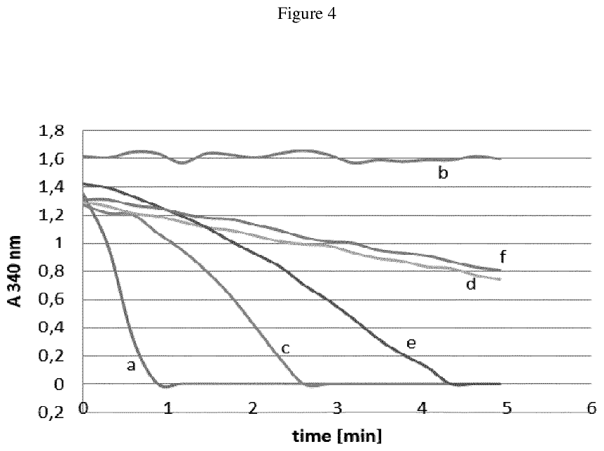

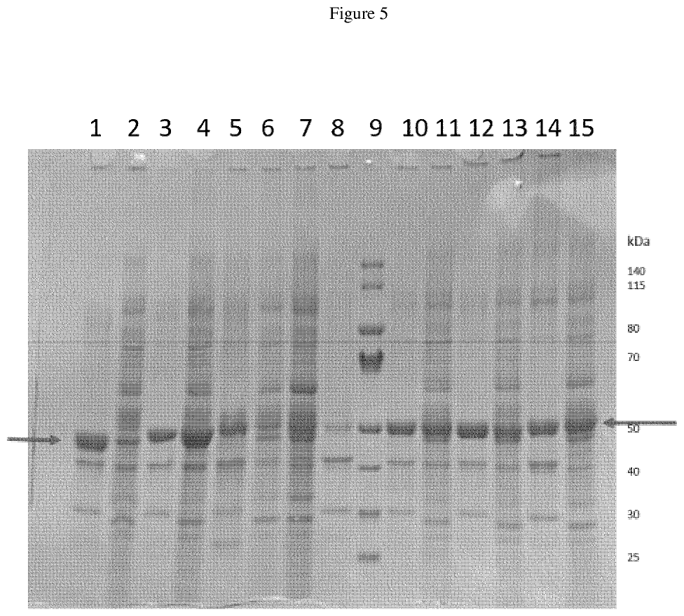

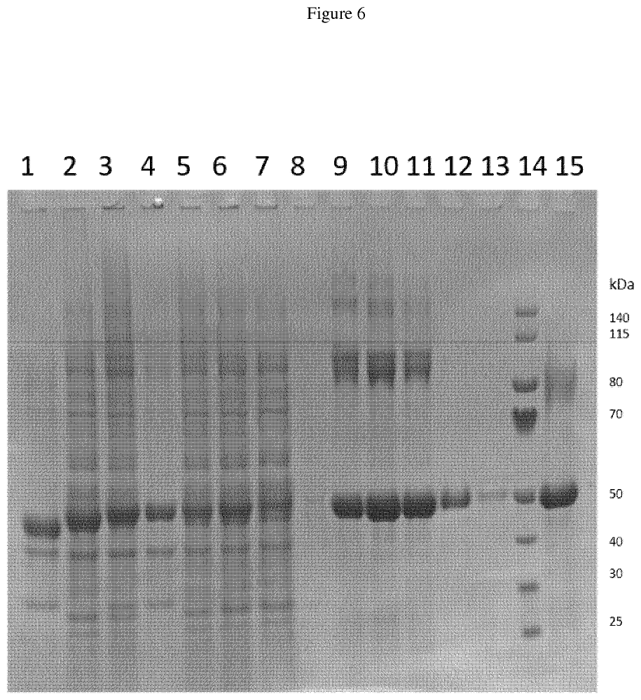

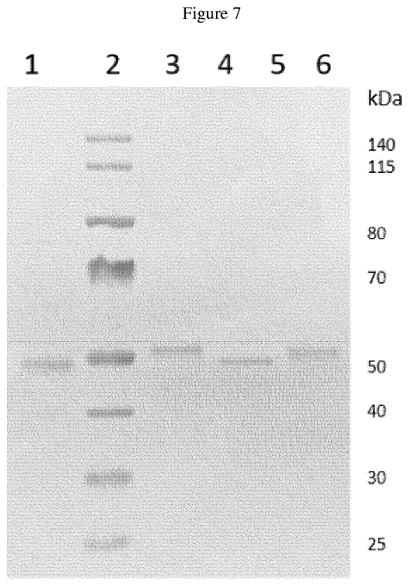

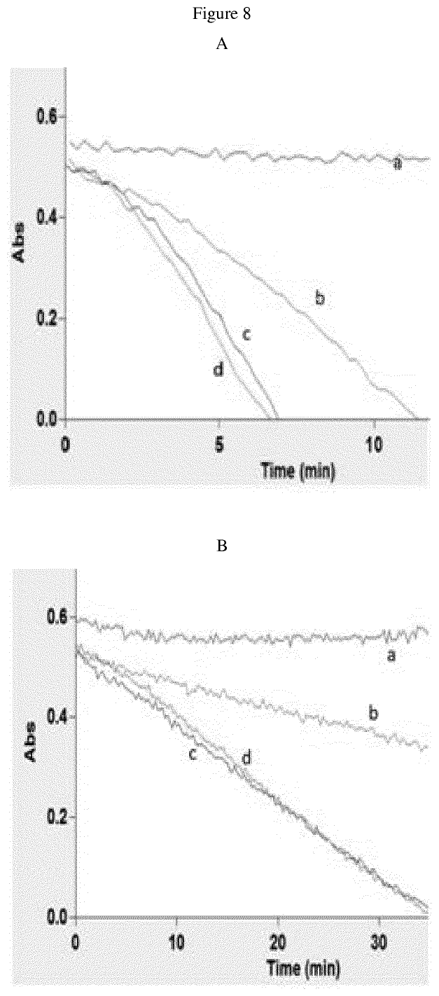

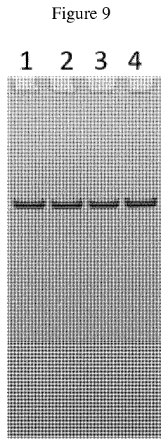

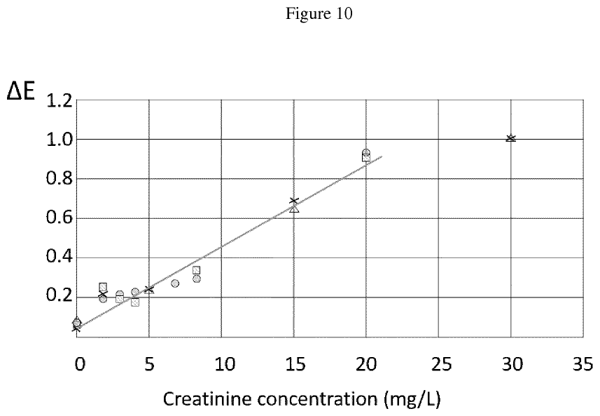

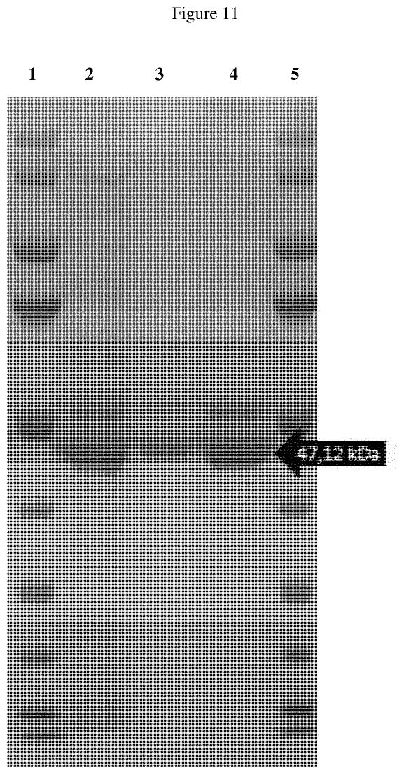

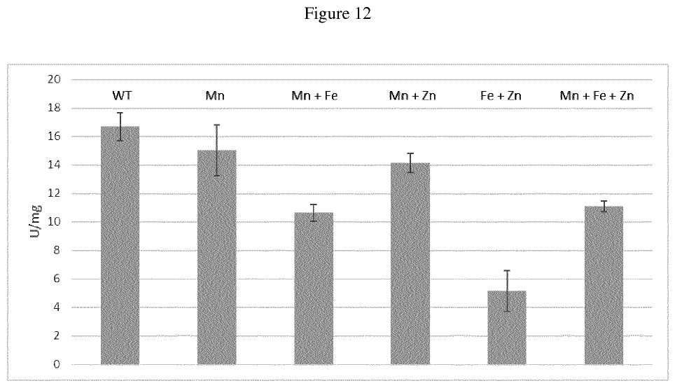

All constructed expression strains were cultivated in 2×TY broth (50 ML in 500 mL Erlenmeyer flasks) supplemented with 100 mg/L ampicillin. Inoculation was performed with an overnight culture (same medium) and cells were grown to an OD between 0.2 and 1.4 at 37° C. Then induction was performed by addition of IPTG at 0.1 mM, and at induction MnCl₂ (0.1 mM final concentration) was also added to the culture. The cultures were then incubated for further 10 h at 25° C. or at 28° C., and the cells harvested by centrifugation. The pellets were either directly resuspended in 50 mM potassium phosphate buffer, pH 7.5 (PPB), or frozen at −20° C. and resuspended after thawing. Cells were disrupted by sonication and lysates were further fractionated for soluble and pellet fractions by centrifugation in 2 steps. In the first step the insoluble material was pelleted at 3,000×g and from the remaining supernatant in the second step insoluble material was pelleted at 16,000×g. The insoluble pellet fractions were resuspended in PPB and all fractions were analyzed for expressed protein by SDS PAGE. Clones containing insert-free vectors were also handled in the same manner as control. SDS PAGE analysis revealed that protein of the expected sizes was produced from the clones of strategy 1 (red arrows). With the clones of strategy 1 of the opposite orientation (control, orientation 2) and the clones of strategy 2, no significant protein band of the expected size was visible (FIG. 2).

When analyzing the soluble and pellet fractions of the clones expressing recombinant creatinine deiminase protein (Strategy 1), it was recognized that the protein derived from clone 1 based on the genomic DNA (SEQ ID NO: 3) is predominantly found in the supernatant fractions indicating well soluble protein. In contrast, the protein derived from the clone based on the DNA according to SEQ ID NO: 2 is nearly totally present in the 3000×g pellet fraction, indicating completely insoluble protein (FIG. 3).

The soluble fractions were also tested for enzymatic activity using an NADH based enzyme-coupled assay. The principle is outlined in the following:

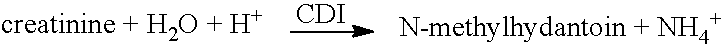

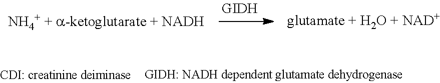

In this assay for determination of creatinine deiminase activity, the consumption of NADH is spectrophotometrically measured at 340 nm. The GlDH (from bovine liver) was obtained from Sigma (Product code G2626). The following reaction setup was used (additions in this order):
0.75 ml creatinine solution (50 mM in 50 mM K-phosphate buffer, pH 7.5)
0.1 ml α-ketoglutarate solution (10 mM in 50 mM K-phosphate buffer, pH 7.5)
8 μL GlDH (about 1 U/μL)
set blank
0.1 mL NADH (3 mM), freshly prepared
0.05 mL creatinine deiminase preparation (if needed diluted in 50 mM K-phosphate buffer, pH 7.5)

The assay is performed at 37° C. Blanking was done before addition of NADH, the reaction was started by addition of the creatinine deiminase preparation. The result of the activity analyses (FIG. 4) showed clearly that the lysate from clone 8 (based on DNA according to SEQ ID NO: 2) did not show activity, only non-specific background as is present with lysates (see control, line d) can be seen. Strong activity could be clearly seen with lysate of the clone 1 (based on DNA according to SEQ ID NO: 3). Weak activity could also be seen with the C-terminally His tagged protein derived from the genomic DNA, though no clear stronger band in the size range of creatinine deiminase could be seen on SDS PAGE (data not shown, but comparable to the his-tagged variants derived from the synthetic DNA, FIG. 2). Using Ni-chelate chromatography, purified protein of this His-tagged clone was readily obtained and showed good activity. The activity results are in accordance with the SDS-PAGE results which showed that the expressed protein from clone 1 (derived from DNA according to SEQ ID NO: 3) is well soluble and thus in an active state, whereas the protein from clone 8 (derived from DNA according to SEQ ID NO: 2) is totally insoluble and thus in a not well folded inactive state. It is also possible that the missing amino acids at the C-terminal end have important function in the enzymatic catalysis mechanism.

### Example 3: Expression of His-Tagged Creatinine Deiminase Based on Clones Containing 5′ and 3′ Up- and Downstream Located Untranslated Genomic Regions of Creatinine Deiminase

From the results of Example 2 it became evident that the 5′ and 3′ up- and downstream located untranslated genomic regions of creatinine deiminase are important for efficient recombinant expression of this enzyme in E. coli. His-tagged proteins allow for efficient purification by Ni-chelate chromatography. Therefore, expression clones for expressing His-tagged variants and containing both, the 5′ and 3′ up- and downstream located untranslated genomic regions of creatinine deiminase were constructed using an overlap extension PCR strategy and primers containing the respective sequences for encoding the amino acids for the His tags. Two variants for each of the N- and C-terminal tags were constructed. One set had only 6 histidines added at the N- or C-terminus. The second set contained a peptide linker and in case of the N-terminal C-tag included a thrombin cleavage site, which would allow removing the tag after purification. All constructs which are summarized in Table 1 were cloned into the vector pBluescript II KS+ in the orientation that transcription can be driven by the inducible lac promoter.

TABLE 1
Summary of constructed His-tagged creatinine deiminase variants. All constructs are
based on the genomic DNA of T. creatinini and the 5’ upstream and 3’ downstream regions
were taken as shown in SEQ ID NO: 3. Short versions contain 6 His codons (CAC or CAT)
after the Met start codon (N-terminal His tag), or 6 His codons (CAC or CAT) before the stop
codon (C-terminal His tag). C-terminal linkers have the sequence Ala Ala Ala Leu Glu (SEQ
ID NO: 5, nucleotide sequence GCGGCCGCACTCGAG, SEQ ID NO: 6) and are inserted
between the creatinine deiminase coding region and the C-terminal His-tag. N-terminal linkers
have the sequence Gly Ser Ser (nucleotide sequence GGCAGCAGC) and are inserted between
the creatinine deiminase 5’-upstream UTR and the N-terminal 6 His codons which are followed
by the peptide sequence (Ser Ser Gly Leu Val Pro Arg Gly Ser His (SEQ ID NO: 7; nucleotide
sequence CACAGCAGCG GCCTGGTGCCGCGCGGCAGCCAT, SEQ ID NO: 8) including
a thrombin cleavage site, and which is fused at its C-terminal end (His) to the creatinine
deiminase coding region.

| | | | 5′- | 3′- |
|---|---|---|---|---|
| Clone # | His tag | Description his tag | upstream | downstream |
| Clone 4h | C terminal, long | His 6 tag, no linker peptide | yes | no |
| Clone 3h | C terminal, short | His 6 tag, no linker peptide | yes | no |
| Clone 1h | C terminal, long | His 6 tag, with linker peptide | yes | yes |
| Clone 6h | C terminal, short | His 6 tag, no linker peptide | yes | yes |
| Clone 11h | N terminal, long | His 6 tag, with linker peptide, | yes | yes |
| | | thrombin cleavage site | | |

The constructed clones were cultivated in shake flask cultures, worked up and analyzed for creatinine deiminase activity in the same manner as described above in Example 2. SDS PAGE analysis (FIG. 5) revealed that the His-tagged variants which contained both the 5′ upstream and the 3′ downstream regions (clones 1h, 6h and 11h) expressed the creatinine deiminase protein in a much better way (larger amounts of soluble creatinine deiminase protein) compared to the variants not containing both regions. The two C-terminally tagged variants not containing the 3′ downstream region were expressed less efficiently and only a smaller amount of soluble creatinine deiminase protein could be seen in the 16,000×g supernatant (clones 3h and 4h), the soluble fraction of these clones showed activity. The His-tagged variants containing both the 5′ and 3′ up- and downstream regions (clones 1h, 6h and 11h) produced larger amounts of soluble protein and the soluble fractions showed good enzymatic activity, but the levels were lower compared to clone 1 correlating to the smaller amount of soluble protein present in the lysates. These results also confirmed that the creatinine deiminase from T. creatinini is well active as C- or N-terminally His tagged variants.

### Example 4: Purification of His-Tagged Variants of Creatinine Deiminase and Determination of Specific Activity

Purification of His-tagged creatinine deiminase was performed according to the following protocol: The fermentation pellet of 50 ml culture was resuspended in 30 ml lysis buffer (50 mM K-phosphate buffer, pH 7.5; 10 mM imidazole 1 mM DTT) and disrupted by sonication. A newly packed column (Ni-NTA-Sepharose™, GE-Healthcare) was loaded with sterile filtered (0.2 μm) lysate (16,000×g supernatant) and subsequently washed with washing buffer (50 mM K-phosphate buffer, pH 7.5; 20 mM imidazole; 1 mM DTI). Protein was then eluted with elution buffer (50 mM K-phosphate buffer, pH 7.5; 250 mM imidazole; 1 mM DTT). The eluted protein was desalted using GE Healthcare 52-1308-00 BB PD-10 desalting columns. The protein was eluted from this column with 50 mM K-phosphate buffer pH7.5, 1 mM DTT according to the manufacturer's protocol. The protein concentration in the final fraction was measured spectrophotometrically with a Nanodrop. The specific extinction coefficient was determined with the ProtPram software. In FIG. 6 the purification of clone 1h is shown as an example.

Determination of specific activity was performed by the activity assay described in Example 2. The two variants clone 1h. The volumetric activity of samples was calculated according to the following equation:

$$U \text{/} ml = \frac{ΔE * {min}^{- 1} * {V}_{total} * dil}{ɛ * {V}_{sample} * d}$$

ε: specific absorption coefficient or NADH at the used wavelength (6.22 ml μmol-¹ cm-¹)
dil: dilution
d: path length of beam in cuvette
Vₜₒₜₐₗ: total volume
Vₛₐₘₚₗₑ: sample volume

Both variants had values for specific activity in the range of up to 40 U/mg protein when 0.5 μg protein per assay was used. Activity values were dependent on the preparation and varied between 20 to 40 U/mg with His-tag purified protein.

### Example 5: Comparison of Creatinine Deiminase Activity of T. creatinini and a Commercially Available Enzyme

Creatinine deiminase originating from a microorganism not specified (Toyobo, product code CNI-311) was purchased. According to patent literature (Toyobo, for example patents from Toyobo JPS61219383A and JP1985000062900) and data on the enzyme, it appears that the enzyme originates from Bacillus subtilis, thus is of different origin than the enzyme derived from T. creatinini. The specification data reports that the enzyme preparation contains 30% of a not specified stabilizer. The purchased lot was declared to have a specific activity of 13.4 U/mg. The enzymatic activity values provided by Toyobo are reported to be determined in a similar manner as described in Example 4, with the difference that with the Toyobo enzyme, a NADPH-dependent GlDH was used.

In order to have a comparable situation, the protein concentrations of the pure enzymes were compared by the band intensities of Coomassie blue stained SDS PAGE gels using highly purified His-tagged creatinine deiminase derived from clone 1h, of which the protein concentration was determined spectrophotometrically by the Nanodrop or the Bradford method (with highly purified protein the same results are obtained). The SDS PAGE for this comparison is shown in FIG. 7.

As can be seen from FIG. 7, the intensity of the Toyobo enzyme was lower and corresponded well to the content of only 70% (30% stabilizer present). Thus, for comparison of the specific activities, the obtained values for the Toyobo enzyme were corrected with this factor.

The activity assays were performed for both enzyme sources, the Toyobo and the T. creatinini derived preparations from clone 1h and clone 11h, using different concentrations of the enzymes. As the reaction is not completely linear over the entire range and slows slightly down at lower NADH concentrations, activity values were determined from regions where the slope was well constant over a longer period (see FIG. 8). The obtained data is shown in Table 2. In order to determine potential cross reactivity to cytosine, creatinine was replaced by cytosine at the same concentration in parallel assays. The result was that with both enzymes the reaction behaved the same as the background control (no enzyme added).

TABLE 2
Determination of the specific activity of the creatinine deiminase preparations. In this
experiment the standard assay using 0.1 mM NADH instead of 0.3 mM NADH was used. Under
these conditions, the linear range was broader. For more details on description of T creatinini
derived preparations and on protein content equilibration see FIG. 7. The corrected activity
values are based on the estimation (see above) of the real enzyme content of the Toyobo enzyme
preparation (70 %).

| | μg | | A₃₄₀ values | | | |
|---|---|---|---|---|---|---|
| | enzyme | Protein | for slope | Slope | Activity | U/mg |
| Enzyme | in assay | mg/ml | range | (ΔE/min) | U/mg | corrected |
| Clone 1 h, purified | 1 | 0.02 | 0.3-0.15 | 0.1004 | 16.1 | — |
| Clone 11 h, purified | 1 | 0.02 | 0.3-0.15 | 0.1005 | 16.2 | — |
| Toyobo | 1 | 0.02 | 0.3-0.15 | 0.0516 | 8.3 | 11.7 |
| Clone l h, purified | 0.25 | 0.005 | 0.4-0.15 | 0.0315 | 20.3 | — |
| Clone 11 h, purified | 0.25 | 0.005 | 0.4-0.15 | 0.0258 | 16.6 | — |
| Toyobo | 0.25 | 0.005 | 0.4-0.15 | 0.0121 | 7.8 | 11.1 |
| Clone l h, purified | 0.1 | 0.002 | 0.4-0.2 | 0.0153 | 24.6 | — |
| Clone 11 h, purified | 0.1 | 0.002 | 0.4-0.2 | 0.017 | 27.3 | — |
| Toyobo | 0.1 | 0.002 | 0.4-0.3 | 0.0048 | 7.7 | 11.0 |

As can be seen from Table 2, the enzyme of the invention is about twice as active as the Toyobo enzyme. Another interesting point is that the enzyme of the invention seems to be more active at lower enzyme concentrations and there is no significant difference in the activity levels of the N- and C-terminally tagged variants.

In FIG. 8, the reaction curves of the activity assays are shown and as can be seen there, higher protein concentrations lead to a retardation of the reaction in the early reaction stage. At the lower protein concentration, also a longer range of linear reaction velocity is observed.

### Example 6: Effect of Bivalent Cations on Enzyme Activity and Fidelity

Clone 1 (see Example 2) encoding the creatinine deiminase of SEQ ID NO: 4 was cultivated and induced for recombinant protein production as described in Example 2. One set of cultures was performed using TB (Terrific Broth) medium supplemented or not supplemented with 0.1 mM Mn⁺⁺. The cells were harvested and disrupted by sonication, and the 16,000×g supernatant was analyzed for creatinine deiminase activity. The obtained results are shown in Table 3.

TABLE 3
Effect of Mn⁺⁺ addition to growth media (0.1% Mn⁺⁺ in the medium) on resulting
specific activity of creatinine deiminase from T. creatinini. Cdi: Enzyme from clone 1, stored
a −20° C. for approximately 1 year (used as reference enzyme). T+ Mn: cultivation in Terrific
Broth with 0.1 mM Mn⁺⁺. TB-Mn: cultivation in Terrific Broth without addition of Mn⁺⁺.

| | | Slope | Activity | Protein | Specific activity |
|---|---|---|---|---|---|
| Sample | Dilution | ΔE/min | U/ml | mg/ml | U/mg |
| TB + Mn | 1:50 | −0.5701 | 92.62 | 25.5 | 3.6 |
| TB − Mn | 1:50 | −0.3509 | 56.41 | 26.6 | 2.1 |

As can be seen from Table 3, addition of Mn++ has a clear positive effect on the specific activity of creatinine deiminase.

In the second set, clone 1h (His-tagged protein) was cultivated in a defined mineral salts medium (standard M9, supplemented with the appropriate amino acids and thiamine) with glucose (10 g/L) as carbon source. A preculture with this medium was inoculated from a master seed lot and used for inoculation of the main cultures. Expression of creatinine deiminase was induced by addition of 0.1 mM IPTG at around OD=1. The cultures were supplemented at the time of induction with the following metal ions (at 0.1 mM): Fe⁺⁺, Mn⁺⁺, Zn⁺⁺ and combinations thereof. The cells were harvested and disrupted, and creatinine deiminase protein was purified by Ni-chelate chromatography from the 16,000×g supernatant. The purified creatinine deiminase protein was examined for activity. As a general trend it could be seen that addition of Mn⁺⁺ and Fe⁺⁺ resulted in active protein but addition of Zn⁺⁺ had a negative effect (based on volumetric activity comparisons).

### Example 7: Metal Analysis of Creatinine Deiminase Protein

Creatinine deiminase from clone 1h (His tagged) was obtained from a standard fermentation using TB medium and addition of 0.1 mM Mn⁺⁺ at the time of induction as described in Example 2. Fe and Zn are present in sufficient amounts in the used complex TB medium and were not supplemented. Mn is not present in the medium in significant amounts and was therefore added. The protein was purified by Ni-chelate chromatography as described in Example 4. A purified preparation having a protein content of 13 mg/ml was subjected to an analysis of metal content by atom absorption spectroscopy (performed at Graz University of Technology, Institute of Analytical Chemistry). The results are shown in Table 4.

TABLE 4
Metal analysis of purified His-tagged creatinine deiminase. In Runs
1 and 2, the same protein preparation was analyzed, Run 2 was
performed after 4 days storage at 4° C.. Only the metals relevant to
be involved in the catalytic activity of the enzyme are shown.

| | | Run 1 | Run 2 | |
|---|---|---|---|---|
| Element, Line | | metal content (mg/kg) | metal content (mg/kg) | |
| Fe | (259.941 nm) | 5.5 ± 0.3 | 5.2 ± 0.3 | |
| Mn | (257.611 nm) | 4.1 ± 0.2 | 3.9 ± 0.2 | |
| Ni | (232.003 nm) | 1.21 ± 0.06 | 1.19 ± 0.06 | m |
| Zn | (213.856 nm) | 3.7 ± 0.2 | 3 ± 0.1 | |

### Example 8: Stability of Creatinine Deiminase from T. creatinini

Purified protein of the clone 1h as described in Example 7 (from cultivation in TB medium and supplemented with 0.1 mM Mn at induction), eluted with 50 mM K-phosphate buffer, pH 7.5, was primarily stored frozen at −20° C. The sample was slowly thawed on ice, diluted to 0.1 μg/μL and then stored for 7 days at 4° C. Then the samples were split and aliquots were stored at 4° C., 23° C. and 37° C. The activity was measured in time intervals. The result was that over 25 days at 4° C. No significant change in activity could be observed under all conditions. The samples from day 25 were also analyzed by SDS PAGE (FIG. 9). With this analysis, also no sign of degradation or decay of protein content was observed.

### Example 9: Quantitative Determination of Creatinine

Purified His-tagged creatinine deiminase (clone 1h) according to SEQ ID NO: 4 as described in Example 7 (from cultivation in TB medium and supplemented with 0.1 mM Mn at induction), was used for quantitative determination of creatinine. The following assay conditions were used:

All solutions: in 50 mM K-phosphate buffer, pH 7.5
Assay setup in the following order:
750 μL Creatinine solution (different concentrations)
100 μL α-ketoglutarate (10 mM)

### 10 μL GlDH

Set blank

### 50 μL NADH (5 mM)

100 μL enzyme solution (3.6 μg/μL; starts the reaction)

The reaction was spectrophotometrically measured at 340 nm and followed over 20 min. The results are shown in FIG. 10, which shows that up to 20 mg/L creatinine, a well linear dependence of the decay of NADH to the creatinine concentration is given. The reaction was complete at 10 min, no significant differences to the ΔE values measured at 20 min are given.

### Example 10: Effect of Addition of Mn²⁺ to the Medium, and of Bivalent Metal Combinations Including Mn²⁺

### Enzyme Preparation

The cultivation of the recombinant E. coli W3110 strain carrying an expression plasmid containing a non-tagged wild-type version of the creatinine deiminase gene and the preparation of cell-free lysate were performed as described in Example 2. The cell lysate was used to purify the protein by ion exchange chromatography using a QFF anion exchange resin column and a ÄKTA chromatography system. The purified protein was finally placed in a 50 mM K-phosphate buffer (pH 7.5) containing 1 mM DDT. The obtained protein preparation (18.7 mg/mL) was analyzed by SDS gel electrophoresis (FIG. 11) and a content of about minimum 75% creatinine deiminase was estimated.

The metal content of this preparation was determined by Optical Atomic Emission Spectrometry with Inductive Coupled Plasma (ICP-OES). The protein sample was therefore set to a concentration of about 9 mg/mL by diluting. The results are summarized in Table 5.

TABLE 5
Results of metal analysis. The molar ratios are based on a molecular
weight of the creatinine deiminase of 47.5 kDa and a content of 75%
creatinine deiminase protein in the tested preparation (FIG. 11), and
given per subunit of protein (the protein contains two subunits).

| Fe (259.941 nm) | 1.6 ± 0.1 mg/kg |
|---|---|
| Mn (257.611 nm) | 0.91 ± 0.04 mg/kg |
| Zn (213.856 nm) | 4.8 ± 0.2 mg/kg |
| Fe (mg/mg Protein) | 0.23 |
| molar ratio Fe:Protein subunit | 0.42 |
| Mn (mg/mg Protein) | 0.13 |
| molar ratio Mn:Protein subunit | 0.25 |
| Zn (mg/mg Protein) | 0.70 |
| molar ratio Zn:Protein subunit | 1.10 |

The obtained protein preparation was used for subsequent metal exchange studies:

Removal of the Metals from the Protein

2 mL of the protein preparation were mixed with 2 mL PDCA dialysis buffer (10 mM 2.6 pyridine carboxylic acid, 66 mM Na-acetate, 20 mM NaCl, pH 5.5). The solution was filled into dialysis tubes (Zellutrans/Roth 6.0) and dialyzed 3 times for 1.5 h in 250 mL PCDA dialysis buffer. A final dialysis step was performed overnight in 1.25 L 50 mM K-phosphate buffer, pH 7.5 followed by filtration through a 0.2 μm membrane filter. The final protein solution (Apo protein) had a concentration of 8.65 mg/mL (4 mL volume).

### Metal Exchange

The Apo protein preparation was diluted to a final volume of 13.3 mL with 50 mM K-phosphate buffer (pH 7.5) containing 1 mM DDT. This resulted in a protein molarity of about 0.05 mM (MW of creatinine deiminase is 47124 Da). Fractions of 1.9 mL were then supplemented to 0.1 mM of the metal ions (each ion, approximately 2 fold molar excess) as indicated in FIG. 12 using 100 mM stock solutions in H₂O of the following metal ions: Mn(II)Cl₂.4×H₂O; Fe(II)SO₄.7×H₂O; Zn(II)Cl₂.

The protein-metal mixtures were incubated at 4° C. for 2.5 h (slightly mixed for several times by shaking the tubes by hand) and afterwards centrifuged in a desk centrifuge (12,000 rpm) to remove possible precipitates (were not observed). The supernatants (1.9 mL each) were then loaded onto PD-10 gel filtration columns which were pre-equilibrated with 50 mM K-phosphate buffer (pH 7.5) containing 1 mM DDT. The column was then spilled with 0.6 mL of the same buffer and the protein finally eluted with 3 mL of the same buffer. The specific activity of all preparations was determined in these solutions. The results are summarized in FIG. 12 and clearly revealed a positive effect of Mn²⁺ alone or in combination with Zn²⁺, Fe²⁺ or both on the activity the enzyme.

1-15. (canceled)

16. A recombinant cell comprising a nucleic acid encoding the creatinine deiminase polypeptide of SEQ ID NO: 4 or a polypeptide having an amino acid sequence at least 95% identical to SEQ ID NO: 4 and having creatinine deiminase activity, wherein the recombinant cell is not a Tissierella creatinini cell.

17. The recombinant cell of claim 16, wherein the nucleic acid further comprises a Tissierella creatinini creatinine deiminase 5′ UTR.

18. The recombinant cell of claim 16, wherein the nucleic acid further comprises a Tissierella creatinini creatinine deiminase 3′ UTR.

19. The recombinant cell of claim 16, wherein the nucleic acid comprises nucleotides 115 to 1374 of SEQ ID NO: 3 or a nucleotide sequence at least 90% identical to positions 115 to 1374 of SEQ ID NO: 3.

20. The recombinant cell of claim 16, wherein the amino acid sequence retains:
(a) the same amino acid found at position 364 of SEQ ID NO: 4 at a position corresponding to position 364 of SEQ ID NO: 4,
(b) the same amino acid found at position 371 of SEQ ID NO: 4 at a position corresponding to position 371 of SEQ ID NO: 4,
(c) the same amino acid found at position 394 of SEQ ID NO: 4 at a position corresponding to position 394 of SEQ ID NO: 4, and/or
(d) one or more, or all of the amino acid residues 410 to 419 of SEQ ID NO: 4 at positions corresponding to positions 410 to 419 of SEQ ID NO: 4.

21. The recombinant cell of claim 16, wherein the creatinine deiminase polypeptide is bound to Mn²⁺.

22. A vector comprising a nucleic acid comprises nucleotides 115 to 1374 of SEQ ID NO: 3 or a nucleotide sequence at least 90% identical to positions 115 to 1374 of SEQ ID NO: 3.

23. A method of producing a recombinant creatinine deiminase polypeptide, the method comprising the steps of:
(i) providing the recombinant cell of claim 16;
(ii) expressing in said cell said nucleic acid encoding the creatinine deiminase polypeptide; and
(iii) isolating the creatinine deiminase polypeptide.

24. A recombinant creatinine deiminase polypeptide obtained by the method of claim 23.

25. The recombinant creatinine deiminase polypeptide of claim 24, wherein the recombinant creatinine deiminase polypeptide further comprises a C-terminal or an N-terminal tag.

26. The recombinant creatinine deiminase polypeptide of claim 25, wherein the tag is an affinity tag, a chromatography tag, an epitope tag, or a fluorescence tag.

27. The recombinant creatinine deiminase polypeptide of claim 26, wherein the tag is selected from the group consisting of AviTag, Calmodulin-tag, polyglutamate tag, E-tag, FLAG-tag, HA-tag, His-tag, Myc-tag, S-tag, SBP-tag, Softag 1, Softag 3, Strep-tag, TC tag, V5 tag, VSV-tag, Xpress tag, Isopeptag, SpyTag, BCCP, Glutathione-S-transferase-tag, Green fluorescent protein-tag, Maltose binding protein-tag, Nus-tag, Thioredoxin-tag, Fc-tag, and Ty tag.

28. The recombinant creatinine deiminase polypeptide of claim 27, wherein the tag is a His-tag.

29. The recombinant creatinine deiminase polypeptide of claim 26, wherein the recombinant creatinine deiminase polypeptide comprises a cleavable peptide linker between the tag and the amino acid sequence of SEQ ID NO: 4 or the amino acid sequence at least 95% identical to SEQ ID NO: 4.

30. The recombinant creatinine deiminase polypeptide of claim 29, wherein the tag is a fluorescence tag.

31. A reagent kit for determining the amount of creatinine in a sample, wherein the kit comprises the recombinant creatinine deiminase polypeptide of claim 24, NADPH or NADH, α-ketoglutarate, glutamate dehydrogenase and optionally a pH-buffer.

32. A sensor suitable for determining the amount of creatinine in a sample, comprising the recombinant creatinine deiminase polypeptide of claim 24 immobilized in or on the sensor.


SEQUENCE LISTING (8 sequences)

SEQ ID NO: 1 - protein, 406 aa, Tissierella creatinini
MMKKFINAKIYKNNEATEILVEDGKIKEIGNNLADCKEVIDLGGKMVTPPYVDPHLHLDYVYTLAELGKTGAGSGTLFEAIEMWPVFKKTLTVESVKKLALKGVMDEVSQGVQHIRTHIDVTDPKFTGLKAMLEMKEELKDIVDIQIVSFPQQGMYTYKGGRELVEEALKMGADVVGGIPHYEPAREYGEMSVKATVELAMKYDKLIDVHCDETDDPQARFIELLNALVYLEGYGAKTSASHTCSFGSADDSYAYRMIDLFKKSKINFISNPTENAYLQGRHDTYPKRRGLTRVKEFMEHGINVAFAQDSINDPWYPMGNGNMMNILDNGIHLAQIMSPQDIEKDLDLITYNGARCLNIQDKYLLEVGKDSNFIVLNGDSPFDVIRNRANVLACVRKGEFYLSKNQ

SEQ ID NO: 2 - DNA, 1593 bp, Tissierella creatinini
ctggcattagtgttattggctatagcaacaattttgtcaataactgataaaaatacattaacaaaagaaaaactgtaagctattaacaatgctaaatttttaaggagtgattttatgatgaaaaagtttattaatgcaaagatttacaagaacaatgaagcaacagaaattttagtagaagacggtaaaatcaaagagattggtaataacttagcagactgtaaagaagtaattgatctaggcggtaaaatggttactccaccttatgtagatcctcacctacatttagattatgtgtatacattggctgaacttggaaaaactggtgctggctcaggaactctttttgaagctattgaaatgtggccagtatttaaaaagactttaactgtagaaagcgttaaaaaacttgctcttaagggggttatggatgaggtttcccaaggggtacaacatattcgtacacatatagatgtaactgatccaaaattcacaggtctaaaagctatgttggaaatgaaagaagaattaaaggacatagttgatatccaaatagtatcattcccacaacaaggaatgtacacatataagggtggacgtgaattagtagaagaagcacttaagatgggtgcagatgttgttggaggaattccgcattatgaaccagctagagaatatggtgaaatgtctgttaaagccacagttgaacttgctatgaaatatgataagctaatagatgttcactgtgatgagacagatgatcctcaagcacgttttattgagctattaaatgcacttgtttatttggaaggttatggtgcaaaaacttcagctagccatacttgttcatttggttcagcagatgattcttatgcatatagaatgatagacttattcaaaaagagcaagataaacttcatctctaatccaactgaaaatgcgtatctacaaggccgtcatgacacttatccaaagcgtcgtggattgactagagttaaagaatttatggagcatggtattaatgttgcatttgcacaagattcaataaacgatccatggtatccaatgggtaacggaaatatgatgaatatacttgacaatggaattcatttagctcaaataatgtcaccacaagatatagaaaaagatttagatttaattacctacaatggtgctcgttgcctaaatatccaagataaatatttattagaagtaggtaaagattcaaactttatcgttcttaacggagacagcccattcgatgtaataagaaaccgtgctaatgttcttgcttgtgttagaaaaggagaattctatttaagcaaaaaccagtagaatatgatgtaaaacttgatttaggcgtaaaatattaatattttaaaataaattccaaattaaccccccggtggtgtaataaactccatcggggggttttttgtgttccagtagaaaataaaaaaatgatataaaaatttagtagtttgaaaaacttaaataaagaaagggcggatttagaatgagtcaaagagacgtattatattcaccagatgcaaagtacaaagataataagggtaaatatggaattgatttagg

SEQ ID NO: 3 - DNA, 1594 bp, Tissierella creatinini
ctggcattagtgttattggctatagcaacaattttgtcaataactgataaaaatacattaacaaaagaaaaactgtaagctattaacaatgctaaatttttaaggagtgattttatgatgaaaaagtttattaatgcaaagatttacaagaacaatgaagcaacagaaattttagtagaagacggtaaaatcaaagagattggtaataacttagcagactgtaaagaagtaattgatctaggcggtaaaatggttactccaccttatgtagatcctcacctacatttagattatgtgtatacattggctgaacttggaaaaactggtgctggctcaggaactctttttgaagctattgaaatgtggccagtatttaaaaagactttaactgtagaaagcgttaaaaaacttgctcttaagggggttatggatgaggtttcccaaggggtacaacatattcgtacacatatagatgtaactgatccaaaattcacaggtctaaaagctatgttggaaatgaaagaagaattaaaggacatagttgatatccaaatagtatcattcccacaacaaggaatgtacacatataagggtggacgtgaattagtagaagaagcacttaagatgggtgcagatgttgttggaggaattccgcattatgaaccagctagagaatatggtgaaatgtctgttaaagccacagttgaacttgctatgaaatatgataagctaatagatgttcactgtgatgagacagatgatcctcaagcacgttttattgagctattaaatgcacttgtttatttggaaggttatggtgcaaaaacttcagctagccatacttgttcatttggttcagcagatgattcatatgcatatagaatgatagacttattcaaaaagagcaagataaacttcatctctaatccaactgaaaatgcgtatctacaaggccgtcatgacacttatccaaagcgtcgtggattgactagagttaaagaatttatggagcatggtattaatgttgcatttgcacaagattcaataaacgatccatggtatccaatgggtaacggaaatatgatgaatatacttgacaatggaattcatttagctcaaataatgtcaccacaagatatagaaaaagatttagatttaattacctacaatggtgctcgttgcctaaatatccaagataaatatggattagaagtaggtaaagatgcaaactttatcgttcttaacggagacagcccattcgatgtaataagaaaccgtgctaatgttcttgcttctgttagaaaaggagaattcctatttaagcaaaaaccagtagaatatgatgtaaaacttgatttaggcgtaaaatattaatattttaaaataaattccaaattaaccccccggtggtgtaataaactccatcggggggttttttgtgttccagtagaaaataaaaaaatgatataaaaatttagtagtttgaaaaacttaaataaagaaagggcggatttagaatgagtcaaagagacgtattatattcaccagatgcaaagtacaaagataataagggtaaatatggaattgatttagg

SEQ ID NO: 4 - protein, 419 aa, Tissierella creatinini
MMKKFINAKIYKNNEATEILVEDGKIKEIGNNLADCKEVIDLGGKMVTPPYVDPHLHLDYVYTLAELGKTGAGSGTLFEAIEMWPVFKKTLTVESVKKLALKGVMDEVSQGVQHIRTHIDVTDPKFTGLKAMLEMKEELKDIVDIQIVSFPQQGMYTYKGGRELVEEALKMGADVVGGIPHYEPAREYGEMSVKATVELAMKYDKLIDVHCDETDDPQARFIELLNALVYLEGYGAKTSASHTCSFGSADDSYAYRMIDLFKKSKINFISNPTENAYLQGRHDTYPKRRGLTRVKEFMEHGINVAFAQDSINDPWYPMGNGNMMNILDNGIHLAQIMSPQDIEKDLDLITYNGARCLNIQDKYGLEVGKDANFIVLNGDSPFDVIRNRANVLASVRKGEFLFKQKPVEYDVKLDLGVKY

SEQ ID NO: 5 - protein, 5 aa, Artificial Sequence
  linker
AAALE

SEQ ID NO: 6 - DNA, 15 bp, Artificial Sequence
  linker
gcggccgcactcgag

SEQ ID NO: 7 - protein, 10 aa, Artificial Sequence
  sequence comprising thrombin cleavage site
SSGLVPRGSH

SEQ ID NO: 8 - DNA, 33 bp, Artificial Sequence
  sequence comprising thrombin cleavage site
cacagcagcggcctggtgccgcgcggcagccat